SEQUENCE LISTING (75 sequences)

SEQ ID NO: 1 - DNA, 642 bp, Synechococcus PCC7942
atgggcgttgagctgcgcagttacgtctacctcgacaatttgcaacggcaacacgcctcctatatcggtacagtcgccacaggctttttgaccctgccaggggatgcctcggtctggattgaaatctccccgggtattgaaatcaaccggatgatggacatcgccctcaaggcggcggtggtgcggcctggagtgcagttcatcgaacgcctctacggcttgatggaagtccacgccagtaatcaaggcgaagtccgtgaagcgggacgtgccgttctctctgctctgggactgacggagcgcgatcgcctcaaacccaaaattgtctccagccaaatcatccgcaatattgatgctcaccaagcgcagctgatcaaccggcagcgccgtggtcaaatgctgctggctggtgaaaccctctacgtcctcgaagtgcaaccggcggcttatgcagcgctagcagccaacgaagcggaaaaggcggcgttgatcaacatcctgcaagtcagtgcgattggcagttttgggcgactctttttgggtggggaggagcgcgacatcattgctggctcgcgggctgctgtagcagcactggaaaacctgtcgggacgtgagcatcccggcgatcgctcgcgggagtag

SEQ ID NO: 2 - protein, 213 aa, Synechococcus PCC7942
MGVELRSYVYLDNLQRQHASYIGTVATGFLTLPGDASVWIEISPGIEINRMMDIALKAAVVRPGVQFIERLYGLMEVHASNQGEVREAGRAVLSALGLTERDRLKPKIVSSQIIRNIDAHQAQLINRQRRGQMLLAGETLYVLEVQPAAYAALAANEAEKAALINILQVSAIGSFGRLFLGGEERDIIAGSRAAVAALENLSGREHPGDRSRE

SEQ ID NO: 3 - DNA, 831 bp, Synechococcus PCC7942
atgtcggcttctcttcccgcctattctcagcctcgcaatgcaggtgcactaggggtcatttgtacccgtagttttccagcggttgtcggcactgcagacatgatgctcaagtcggccgatgtcacattgatcggctatgagaaaacaggctcgggcttttgtacagcaatcatccggggtggctatgccgacatcaagctggctcttgaggctggcgtagcgacagctcgtcagtttgagcagtacgtttccagcactattctgccgcggcctcaaggcaacctcgaagccgtgttgccgattagccggcggctctcccaagaagccatggccacgcgatcgcatcagaatgttggcgcgattgggctaattgagaccaatgggttccctgctttggttggagcagccgatgccatgctcaaatcggctaacgtcaagctgatttgttatgagaaaacgggcagcggtctctgtactgcgatcgtgcaaggcacggtttctaatgtgaccgttgcggtcgaagccgggatgtatgccgctgagcggatcggccagctcaacgcaatcatggtcattcccagaccgctagacgacttgatggacagcttgcctgagccgcagtcggatagcgaagcagcccagccactccaattaccgctgcgggttcgcgaaaaacaaccgctgttggagctaccggaactcgaacggcagccgatcgcgatcgaagcaccgcgacttttagcagaagagcgacagtctgcgttggaattggctcaagagacaccgctcgccgagcccttagagctccccaatcctcgtgatgatcagtga

SEQ ID NO: 4 - protein, 276 aa, Synechococcus PCC7942
MSASLPAYSQPRNAGALGVICTRSFPAVVGTADMMLKSADVTLIGYEKTGSGFCTAIIRGGYADIKLALEAGVATARQFEQYVSSTILPRPQGNLEAVLPISRRLSQEAMATRSHQNVGAIGLIETNGFPALVGAADAMLKSANVKLICYEKTGSGLCTAIVQGTVSNVTVAVEAGMYAAERIGQLNAIMVIPRPLDDLMDSLPEPQSDSEAAQPLQLPLRVREKQPLLELPELERQPIAIEAPRLLAEERQSALELAQETPLAEPLELPNPRDDQ

SEQ ID NO: 5 - DNA, 309 bp, Synechococcus PCC7942
atgcctattgcggttggaatgatcgagaccctgggcttcccggctgttgtggaagcagctgacgcgatggtcaaagcagcgcgtgtcacgctggttggctatgagaagattggcagcggccgcgtcactgtcattgtccggggagacgtttcggaagttcaagcttctgtctctgcgggtctcgattcggcgaaacgggttgccggtggtgaagtgctgtcgcaccacatcattgcgcgtccccacgagaacttggaatacgttctcccgattcgctacaccgaagctgttgaacaattccgcatgtaa

SEQ ID NO: 6 - protein, 102 aa, Synechococcus PCC7942
MPIAVGMIETLGFPAVVEAADAMVKAARVTLVGYEKIGSGRVTVIVRGDVSEVQASVSAGLDSAKRVAGGEVLSHHIIARPHENLEYVLPIRYTEAVEQFRM

SEQ ID NO: 7 - DNA, 300 bp, Synechococcus PCC7942
atgcgcattgctaaggttcgcggaaccgtagtcagtacctacaaagagcccagcctgcaaggggtaaagttcttggttgttcagttcttggatgaggctggacaggcacttcaagagtatgaggttgctgctgacatgattggcgctggcgttgacgagtgggtgttgattagccgcggcagtcaagcgcgccatgtgcgcgattgtcaggaacgaccggttgatgcagctgtcattgccatcatcgatacggtcaacgtggaaaaccgctccgtctacgacaaacgcgagcacagctaa

SEQ ID NO: 8 - protein, 99 aa, Synechococcus PCC7942
MRIAKVRGTVVSTYKEPSLQGVKFLVVQFLDEAGQALQEYEVAADMIGAGVDEWVLISRGSQARHVRDCQERPVDAAVIAIIDTVNVENRSVYDKREHS

SEQ ID NO: 9 - DNA, 972 bp, Artificial Sequence
  Synthetic Construct
tctgcttataacggacaaggtcgattaagttctgaagtaattactcaagttcgaagtttgttaaaccaaggatatcgaattggaactgaacatgctgataagagacgatttagaactagttcttggcaaccttgtgctcctattcaatctactaatgagagacaggtattgtctgaacttgaaaattgtctttctgaacatgaaggtgaatacgttcgattgttaggaattgataccaatactagatctcgtgtttttgaagctttaattcaacgacctgatggttcggttcctgaatcgttaggatctcaacctgtggcagtagcttcaggtggaggtcgacaatcatcttatgcaagtgtatctggaaatttatctgctgaagtagttaataaagtacgtaatctattagctcaaggatatcgaattggtacagaacacgcagacaaaagacgatttcgtacttcttcatggcagtcatgcgcaccaatccagagttctaacgagcgtcaagttcttgctgagcttgaaaactgcttaagtgagcatgagggagagtacgttagattacttggtatcgatactgcttctagaagtcgtgttttcgaagcacttatacaagatccacaaggacctgtaggttctgctaaagctgcagccgctcctgtatcttcagctactccaagttctcatagttatacttctaatggatctagttcgagcgatgtcgctggacaggttcgaggtcttctagcacagggttaccgtataagtgctgaagtagctgataagcgtagattccaaacaagttcttggcaaagtttacctgctcttagtggacagtctgaagcaactgtattgcctgctttggagtcaattcttcaagaacacaaaggtaagtatgtacgtcttattgggattgaccctgcagctcgtcgtcgagtagctgagttgttgattcagaagccgtaa

SEQ ID NO: 10 - protein, 323 aa, Synechococcus PCC7942
SAYNGQGRLSSEVITQVRSLLNQGYRIGTEHADKRRFRTSSWQPCAPIQSTNERQVLSELENCLSEHEGEYVRLLGIDTNTRSRVFEALIQRPDGSVPESLGSQPVAVASGGGRQSSYASVSGNLSAEVVNKVRNLLAQGYRIGTEHADKRRFRTSSWQSCAPIQSSNERQVLAELENCLSEHEGEYVRLLGIDTASRSRVFEALIQDPQGPVGSAKAAAAPVSSATPSSHSYTSNGSSSSDVAGQVRGLLAQGYRISAEVADKRRFQTSSWQSLPALSGQSEATVLPALESILQEHKGKYVRLIGIDPAARRRVAELLIQKP

SEQ ID NO: 11 - DNA, 1614 bp, Artificial Sequence
  Synthetic Construct
ccttctccaacaactgtacctgttgctactgctggtagattggctgaaccttatattgatcctgctgctcaagttcatgcaattgctagtataatcggcgacgtacgtattgcagctggagtaagagttgcagctggagtttcgattcgtgctgatgaaggagcaccatttcaagtaggtaaagaatctattcttcaagagggagctgtaattcatggattggaatatggtcgtgtattgggtgatgatcaagcagactattccgtctggataggccagcgagtagctattactcataaagcacttattcatggaccagcttatcttggagatgattgttttgtaggtttccgatctaccgtatttaacgctcgtgttggagccggttcggtaatcatgatgcacgcccttgtccaagacgtagagattcctcctggtagatatgttccttctggagcaattatcacaactcaacagcaagctgatcgactacctgaagttcgtcctgaagatcgagaatttgctagacatataattggatcacctccagtaattgtaagatctactccagcagctactgctgattttcactcaacaccaactccttctccacttcgtccatcgtctagtgaggcaacaactgtatctgcttataacggacaaggtcgattaagttctgaagtaattactcaagttcgaagtttgttaaaccaaggatatcgaattggaactgaacatgctgataagagacgatttagaactagttcttggcaaccttgtgctcctattcaatctactaatgagagacaggtattgtctgaacttgaaaattgtctttctgaacatgaaggtgaatacgttcgattgttaggaattgataccaatactagatctcgtgtttttgaagctttaattcaacgacctgatggttcggttcctgaatcgttaggatctcaacctgtggcagtagcttcaggtggaggtcgacaatcatcttatgcaagtgtatctggaaatttatctgctgaagtagttaataaagtacgtaatctattagctcaaggatatcgaattggtacagaacacgcagacaaaagacgatttcgtacttcttcatggcagtcatgcgcaccaatccagagttctaacgagcgtcaagttcttgctgagcttgaaaactgcttaagtgagcatgagggagagtacgttagattacttggtatcgatactgcttctagaagtcgtgttttcgaagcacttatacaagatccacaaggacctgtaggttctgctaaagctgcagccgctcctgtatcttcagctactccaagttctcatagttatacttctaatggatctagttcgagcgatgtcgctggacaggttcgaggtcttctagcacagggttaccgtataagtgctgaagtagctgataagcgtagattccaaacaagttcttggcaaagtttacctgctcttagtggacagtctgaagcaactgtattgcctgctttggagtcaattcttcaagaacacaaaggtaagtatgtacgtcttattgggattgaccctgcagctcgtcgtcgagtagctgagttgttgattcagaagccg

SEQ ID NO: 12 - protein, 538 aa, Synechococcus PCC7942
PSPTTVPVATAGRLAEPYIDPAAQVHAIASIIGDVRIAAGVRVAAGVSIRADEGAPFQVGKESILQEGAVIHGLEYGRVLGDDQADYSVWIGQRVAITHKALIHGPAYLGDDCFVGFRSTVFNARVGAGSVIMMHALVQDVEIPPGRYVPSGAIITTQQQADRLPEVRPEDREFARHIIGSPPVIVRSTPAATADFHSTPTPSPLRPSSSEATTVSAYNGQGRLSSEVITQVRSLLNQGYRIGTEHADKRRFRTSSWQPCAPIQSTNERQVLSELENCLSEHEGEYVRLLGIDTNTRSRVFEALIQRPDGSVPESLGSQPVAVASGGGRQSSYASVSGNLSAEVVNKVRNLLAQGYRIGTEHADKRRFRTSSWQSCAPIQSSNERQVLAELENCLSEHEGEYVRLLGIDTASRSRVFEALIQDPQGPVGSAKAAAAPVSSATPSSHSYTSNGSSSSDVAGQVRGLLAQGYRISAEVADKRRFQTSSWQSLPALSGQSEATVLPALESILQEHKGKYVRLIGIDPAARRRVAELLIQKP

SEQ ID NO: 13 - DNA, 1419 bp, Synechococcus PCC7942
atgcctaaaacccaaagtgctgctggatataaagcaggagttaaagattataaacttacctattatactccagattatactccaaaagataccgatttacttgctgcatttcgattcagtcctcaaccaggagtaccagcagatgaagctggtgctgcaattgcagcagaaagttcaacaggaacttggactaccgtttggacagatcttctaaccgatatggatagatataaagggaaatgttatcatattgaaccagtacaaggagaagagaattcctattttgcttttattgcatatcctcttgatttgtttgaagaaggatcagttactaacattctaactagtatcgttggaaatgtatttggattcaaagctatacgatcactacgtttggaagatatacgtttcccagttgctttggttaaaactttccaagggcctccacatggaattcaagttgaaagagatttattaaacaagtatgggcgaccaatgcttggatgtacaattaagcctaaattagggctatctgctaaaaactatggacgtgctgtatatgagtgtttaagaggaggattagattttactaaagatgatgaaaatattaattcacaaccttttcaacgatggcgagatagatttctttttgttgccgatgccattcataaatcacaagccgagactggagaaattaagggacattatctaaatgtaaccgccccaacatgtgaagaaatgatgaagcgagctgaatttgctaaagaattgggtatgccaatcataatgcatgattttctaactgctggattcaccgccaatactactttagctaagtggtgtcgtgataatggtgtattacttcatatacatcgagcaatgcatgctgtaatagatagacaacgaaaccatggtattcattttcgtgttttagcaaaatgtcttcgattgagtggaggggatcatttgcattctgggactgttgtagggaaattggaaggagataaagcctcaactcttggatttgtagatctaatgcgagaagatcatatagaggcagatagaagtagaggtgtatttttcacccaagattgggctagtatgcctggggttcttcctgtagctagtggaggaattcatgtttggcacatgccagcactagtagaaatcttcggagatgattcagttttacaatttggtggaggaactctaggtcatccatggggaaatgcaccaggtgcaacagcaaatcgtgttgctttagaagcatgtgtacaagctcgtaatgagggtcgagatttatatagagaagggggagatatacttagagaggctggaaaatggtctccagaattggcagctgcccttgatctatggaaagaaataaagtttgaatttgagacaatggataagctttaa

SEQ ID NO: 14 - protein, 472 aa, Synechococcus PCC7942
MPKTQSAAGYKAGVKDYKLTYYTPDYTPKDTDLLAAFRFSPQPGVPADEAGAAIAAESSTGTWTTVWTDLLTDMDRYKGKCYHIEPVQGEENSYFAFIAYPLDLFEEGSVTNILTSIVGNVFGFKAIRSLRLEDIRFPVALVKTFQGPPHGIQVERDLLNKYGRPMLGCTIKPKLGLSAKNYGRAVYECLRGGLDFTKDDENINSQPFQRWRDRFLFVADAIHKSQAETGEIKGHYLNVTAPTCEEMMKRAEFAKELGMPIIMHDFLTAGFTANTTLAKWCRDNGVLLHIHRAMHAVIDRQRNHGIHFRVLAKCLRLSGGDHLHSGTVVGKLEGDKASTLGFVDLMREDHIEADRSRGVFFTQDWASMPGVLPVASGGIHVWHMPALVEIFGDDSVLQFGGGTLGHPWGNAPGATANRVALEACVQARNEGRDLYREGGDILREAGKWSPELAAALDLWKEIKFEFETMDKL

SEQ ID NO: 15 - DNA, 336 bp, Artificial Sequence
  Synthetic Construct
atgagtatgaaaaccttgccaaaagaacgaagatttgaaaccttcagttatttacctcctctttctgatcgtcaaattgctgctcaaatcgaatatatgatagaacaaggttttcatccattgatagaatttaatgaacattcaaatccagaagaattctattggaccatgtggaaactacctttgtttgattgtaagtctccacaacaagtattggatgaggtacgagaatgtcgttccgagtatggtgattgttatattagagttgcaggatttgataacatcaaacaatgtcaaaccgttagtttcattgtgcatcgacctggaagatattaa

SEQ ID NO: 16 - protein, 111 aa, Synechococcus PCC7942
MSMKTLPKERRFETFSYLPPLSDRQIAAQIEYMIEQGFHPLIEFNEHSNPEEFYWTMWKLPLFDCKSPQQVLDEVRECRSEYGDCYIRVAGFDNIKQCQTVSFIVHRPGRY

SEQ ID NO: 17 - DNA, 459 bp, Artificial Sequence
  Synthetic Construct
atggcgtcaacgcagagggcgaagccgatggagatgccccgcatcagtcgcgatacagcccgcatgttggtcaattacctgacctatcaagcggtctgtgtgattcgggatcaattggctgagacgaatccggccggtgcataccggctgcaggttttctcggctgagttctcctttcaggatggagaagcttacctagcagctctactcaaccacgatcgcgaattgggcctgcgggtgatgacagtacgggaacatttggccgagcatattctcgactacctgccggagatgacgatcgctcagatccaggaggcgaatattaatcatcgccgtgctttgcttgaacggctgacgggtcttggggcagagcctagcttgccggagaccgaggtgagcgatcgccccagtgactcagccactcctgatgatgcttctaatgcctcccatgcggattga

SEQ ID NO: 18 - protein, 152 aa, Synechococcus PCC7942
MASTQRAKPMEMPRISRDTARMLVNYLTYQAVCVIRDQLAETNPAGAYRLQVFSAEFSFQDGEAYLAALLNHDRELGLRVMTVREHLAEHILDYLPEMTIAQIQEANINHRRALLERLTGLGAEPSLPETEVSDRPSDSATPDDASNASHAD

SEQ ID NO: 19 - DNA, 975 bp, Synechococcus PCC7942
atgagcgcttataacggccaaggccgactcagttccgaagtcatcacccaagtccggagtttgctgaaccagggctatcggattgggacggaacatgcggacaagcgccgcttccggactagctcttggcagccctgcgcgccgattcaaagcacgaacgagcgccaggtcttgagcgaactggaaaattgtctgagcgaacacgaaggtgaatacgttcgcttgctcggcatcgataccaatactcgcagccgtgtttttgaagccctgattcaacggcccgatggttcggttcctgaatcgctggggagccaaccggtggcagtcgcttccggtggtggccgtcagagcagctatgccagcgtcagcggcaacctctcagcagaagtggtcaataaagtccgcaacctcttagcccaaggctatcggattgggacggaacatgcagacaagcgccgctttcggactagctcttggcagtcctgcgcaccgattcaaagttcgaatgagcgccaggttctggctgaactggaaaactgtctgagcgagcacgaaggtgagtacgttcgcctgctgggcatcgacactgctagccgcagtcgtgtttttgaagccctgatccaagatccccaaggaccggtgggttccgccaaagctgccgccgcacctgtgagttcggcaacgcccagcagccacagctacacctcaaatggatcgagttcgagcgatgtcgctggacaggttcggggtctgctagcccaaggctaccggatcagtgcggaagtcgccgataagcgtcgcttccaaaccagctcttggcagagtttgccggctctgagtggccagagcgaagcaactgtcttgcctgctttggagtcaattctgcaagagcacaagggtaagtatgtgcgcctgattgggattgaccctgcggctcgtcgtcgcgtggctgaactgttgattcaaaagccgtaa

SEQ ID NO: 20 - protein, 324 aa, Synechococcus PCC7942
MSAYNGQGRLSSEVITQVRSLLNQGYRIGTEHADKRRFRTSSWQPCAPIQSTNERQVLSELENCLSEHEGEYVRLLGIDTNTRSRVFEALIQRPDGSVPESLGSQPVAVASGGGRQSSYASVSGNLSAEVVNKVRNLLAQGYRIGTEHADKRRFRTSSWQSCAPIQSSNERQVLAELENCLSEHEGEYVRLLGIDTASRSRVFEALIQDPQGPVGSAKAAAAPVSSATPSSHSYTSNGSSSSDVAGQVRGLLAQGYRISAEVADKRRFQTSSWQSLPALSGQSEATVLPALESILQEHKGKYVRLIGIDPAARRRVAELLIQKP

SEQ ID NO: 21 - DNA, 309 bp, Synechococcus PCC7942
atgccaattgctgtcggaacgattcaaaccctcggatttccgccgattattgctgcggcagatgcgatggtcaaagcggctcgggtcaccatcacccagtatggattggcggaaagtgcccaattctttgtctcggtgcggggacctgtttcggaagtcgaaacggctgttgaagcagggttgaaagcagttgctgaaaccgaaggggcagagctgatcaattacatcgtcatcccgaatccacaagaaaacgtggaaacggtgatgccgatcgacttcacggctgaatccgagccctttcggtcttaa

SEQ ID NO: 22 - protein, 102 aa, Synechococcus PCC7942
MPIAVGTIQTLGFPPIIAAADAMVKAARVTITQYGLAESAQFFVSVRGPVSEVETAVEAGLKAVAETEGAELINYIVIPNPQENVETVMPIDFTAESEPFRS

SEQ ID NO: 23 - DNA, 342 bp, Synechococcus PCC7942
atgtctcagcaggcaattggctcgctggaaacgaagggctttcccccaatcttggcggcagctgatgccatggtcaaagctggccgaatcacgattgtgagctacatgcgggccggtagcgctcgctttgcagtcaacattcggggggatgtctcagaagtcaaaacggcgatggatgcgggcattgaagccgcgaaaaatacgcctggtggcaccctcgaaacgtgggtgatcatccctcgcccgcatgaaaacgtggaagcggtcttcccgatcggctttggcccagaagtggaacaatatcgactctctgccgaaggaactggtagtggccgccgttaa

SEQ ID NO: 24 - protein, 113 aa, Synechococcus PCC7942
MSQQAIGSLETKGFPPILAAADAMVKAGRITIVSYMRAGSARFAVNIRGDVSEVKTAMDAGIEAAKNTPGGTLETWVIIPRPHENVEAVFPIGFGPEVEQYRLSAEGTGSGRR

SEQ ID NO: 25 - DNA, 819 bp, Synechococcus PCC7942
atgcgcaagctcatcgaggggttacggcatttccgtacgtcctactacccgtctcatcgggacctgttcgagcagtttgccaaaggtcagcaccctcgagtcctgttcattacctgctcagactcgcgcattgaccctaacctcattacccagtcgggcatgggtgagctgttcgtcattcgcaacgctggcaatctgatcccgcccttcggtgccgccaacggtggtgaaggggcatcgatcgaatacgcgatcgcagctttgaacattgagcatgttgtggtctgcggtcactcgcactgcggtgcgatgaaagggctgctcaagctcaatcagctgcaagaggacatgccgctggtctatgactggctgcagcatgcccaagccacccgccgcctagtcttggataactacagcggttatgagactgacgacttggtagagattctggtcgccgagaatgtgctgacgcagatcgagaaccttaagacctacccgatcgtgcgatcgcgccttttccaaggcaagctgcagatttttggctggatttatgaagttgaaagcggcgaggtcttgcagattagccgtaccagcagtgatgacacaggcattgatgaatgtccagtgcgtttgcccggcagccaggagaaagccattctcggtcgttgtgtcgtccccctgaccgaagaagtggccgttgctccaccagagccggagcctgtgatcgcggctgtggcggctccacccgccaactactccagtcgcggttggttggcccctgaacaacaacagcggatttatcgcggcaatgctagctag

SEQ ID NO: 26 - protein, 272 aa, Synechococcus PCC7942
MRKLIEGLRHFRTSYYPSHRDLFEQFAKGQHPRVLFITCSDSRIDPNLITQSGMGELFVIRNAGNLIPPFGAANGGEGASIEYAIAALNIEHVVVCGHSHCGAMKGLLKLNQLQEDMPLVYDWLQHAQATRRLVLDNYSGYETDDLVEILVAENVLTQIENLKTYPIVRSRLFQGKLQIFGWIYEVESGEVLQISRTSSDDTGIDECPVRLPGSQEKAILGRCVVPLTEEVAVAPPEPEPVIAAVAAPPANYSSRGWLAPEQQQRIYRGNAS

SEQ ID NO: 27 - DNA, 486 bp, Artificial Sequence
  Synthetic Construct
atgcacctaccacctctggaacctccaattagtgatcgatattttgcttcaggtgaggttacaattgcagctgatgtagttatagcacctggggtattgcttattgcagaagctgacagtcggattgaaattgcatcaggagtttgtattggactcggcagtgtaattcatgcacgaggaggtgcaattataattcaagcaggcgctttactggcagctggcgtacttattgttggacaatcaattgttgggcggcaagcatgtcttggtgcatccactacccttgttaatacttctattgaggctggaggtgttacagcaccaggaagtttactttcagctgaaacacctcccacgactgctacagttagttcctcagagcctgctgggaggtctccccaatcctcagcaattgctcatcctaccaaagtatatggaaaagaacaatttttaaggatgcgacaaagtatgttccctgatcgataa

SEQ ID NO: 28 - protein, 161 aa, Synechococcus PCC7942
MHLPPLEPPISDRYFASGEVTIAADVVIAPGVLLIAEADSRIEIASGVCIGLGSVIHARGGAIIIQAGALLAAGVLIVGQSIVGRQACLGASTTLVNTSIEAGGVTAPGSLLSAETPPTTATVSSSEPAGRSPQSSAIAHPTKVYGKEQFLRMRQSMFPDR

SEQ ID NO: 29 - protein, 17 aa, Artificial Sequence
  Synthetic Construct
YGKEQFLRMRQSMFPDR

SEQ ID NO: 30 - protein, 9 aa, Artificial Sequence
  Synthetic Construct
GGSGGSGGS

SEQ ID NO: 31 - DNA, 195 bp, Artificial Sequence
  Synthetic Construct
atggattcacagctagtcttgtctctgaagctgaatccaagcttcactcctctttctcctctcttccctttcactccatgttcttctttttcgccgtcgctccggttttcttcttgctactcccgccgcctctattctccggttaccgtctacgccgcgaagaaactctcccacaaaatcagttctgaattcgat

SEQ ID NO: 32 - DNA, 51 bp, Artificial Sequence
  Synthetic Construct
tacggcaaggaacagtttttgcggatgcgccagagcatgttccccgatcgc

SEQ ID NO: 33 - DNA, 27 bp, Artificial Sequence
  Synthetic Construct
ggaggttctggtggaagtgggggttca

SEQ ID NO: 34 - DNA, 27 bp, Artificial Sequence
  Synthetic Construct
atggattcacagctagtcttgtctctg

SEQ ID NO: 35 - DNA, 34 bp, Artificial Sequence
  Synthetic Construct
gtcgcgatcgaattcagaactgattttgtgggag

SEQ ID NO: 36 - DNA, 52 bp, Artificial Sequence
  Synthetic Construct
caaaatcagttctgaattcgatcgcgacatgcctattgcggttggaatgatc

SEQ ID NO: 37 - DNA, 24 bp, Artificial Sequence
  Synthetic Construct
catgcggaattgttcaacagcttc

SEQ ID NO: 38 - DNA, 48 bp, Artificial Sequence
  Synthetic Construct
caaaatcagttctgaattcgatcgcgacatgcgcattgctaaggttcg

SEQ ID NO: 39 - DNA, 18 bp, Artificial Sequence
  Synthetic Construct
gctgtgctcgcgtttgtc

SEQ ID NO: 40 - DNA, 50 bp, Artificial Sequence
  Synthetic Construct
caaaatcagttctgaattcgatcgcgacatgtcggcttctcttcccgcct

SEQ ID NO: 41 - DNA, 24 bp, Artificial Sequence
  Synthetic Construct
ctgatcatcacgaggattggggag

SEQ ID NO: 42 - DNA, 42 bp, Artificial Sequence
  Synthetic Construct
agttctgaattcgatcgcgacccttctccaacaactgtacct

SEQ ID NO: 43 - DNA, 22 bp, Artificial Sequence
  Synthetic Construct
cggcttctgaatcaacaactca

SEQ ID NO: 44 - DNA, 50 bp, Artificial Sequence
  Synthetic Construct
caaaatcagttctgaattcgatcgcgacatgcacctaccacctctggaac

SEQ ID NO: 45 - DNA, 24 bp, Artificial Sequence
  Synthetic Construct
tcgatcagggaacatactttgtcg

SEQ ID NO: 46 - DNA, 53 bp, Artificial Sequence
  Synthetic Construct
caaaatcagttctgaattcgatcgcgactacggcaaggaacagtttttgcgga

SEQ ID NO: 47 - DNA, 23 bp, Artificial Sequence
  Synthetic Construct
gaccttagtggggtgggcgattg

SEQ ID NO: 48 - DNA, 57 bp, Artificial Sequence
  Synthetic Construct
caaaatcagttctgaattcgatcgcgacatgcctattgctgtaggtactatacaaac

SEQ ID NO: 49 - DNA, 24 bp, Artificial Sequence
  Synthetic Construct
agatcggaatggctcagattcagc

SEQ ID NO: 50 - DNA, 56 bp, Artificial Sequence
  Synthetic Construct
caaaatcagttctgaattcgatcgcgacatgtctcaacaagctattggatctcttg

SEQ ID NO: 51 - DNA, 24 bp, Artificial Sequence
  Synthetic Construct
acgtcgaccactaccagttccttc

SEQ ID NO: 52 - DNA, 59 bp, Artificial Sequence
  Synthetic Construct
caaaatcagttctgaattcgatcgcgacatggttagtaaaggtgaagaattgtttactg

SEQ ID NO: 53 - DNA, 26 bp, Artificial Sequence
  Synthetic Construct
cttgtacaactcgtccattcctaaag

SEQ ID NO: 54 - DNA, 49 bp, Artificial Sequence
  Synthetic Construct
atgcacggcgcgcctcacacgcgtcgacgagtcagtaataaacggcgtc

SEQ ID NO: 55 - DNA, 58 bp, Artificial Sequence
  Synthetic Construct
actagttctagaggatcccgcactcgagtgttaatcagaaaaactcagattaatcgac

SEQ ID NO: 56 - DNA, 43 bp, Artificial Sequence
  Synthetic Construct
atgcacggcgcgcctcacacgcgtgcacgccccagagcttctc

SEQ ID NO: 57 - DNA, 25 bp, Artificial Sequence
  Synthetic Construct
gctagagtcgatttggtgtatcgag

SEQ ID NO: 58 - DNA, 25 bp, Artificial Sequence
  Synthetic Construct
gagctctagagtccgcaaaaatcac

SEQ ID NO: 59 - DNA, 51 bp, Artificial Sequence
  Synthetic Construct
atgcacgtttaaacggcgcgccggtcactggattttggttttaggaattag

SEQ ID NO: 60 - DNA, 58 bp, Artificial Sequence
  Synthetic Construct
gtcgattaatctgagtttttctgattaacagatggattcacagctagtcttgtctctg

SEQ ID NO: 61 - DNA, 52 bp, Artificial Sequence
  Synthetic Construct
ctcgatacaccaaatcgactctagcatggattcacagctagtcttgtctctg

SEQ ID NO: 62 - DNA, 52 bp, Artificial Sequence
  Synthetic Construct
gtgatttttgcggactctagagctcttacatgcggaattgttcaacagcttc

SEQ ID NO: 63 - DNA, 50 bp, Artificial Sequence
  Synthetic Construct
gtgatttttgcggactctagagctcttagctgtgctcgcgtttgtcgtag

SEQ ID NO: 64 - DNA, 52 bp, Artificial Sequence
  Synthetic Construct
gtgatttttgcggactctagagctcttactgatcatcacgaggattggggag

SEQ ID NO: 65 - DNA, 52 bp, Artificial Sequence
  Synthetic Construct
gtgatttttgcggactctagagctcttacggcttctgaatcaacaactcagc

SEQ ID NO: 66 - DNA, 56 bp, Artificial Sequence
  Synthetic Construct
gtgatttttgcggactctagagctcttatcgatcagggaacatactttgtcgcatc

SEQ ID NO: 67 - DNA, 52 bp, Artificial Sequence
  Synthetic Construct
gtgatttttgcggactctagagctcttaagatcggaatggctcagattcagc

SEQ ID NO: 68 - DNA, 52 bp, Artificial Sequence
  Synthetic Construct
gtgatttttgcggactctagagctcttaacgtcgaccactaccagttccttc

SEQ ID NO: 69 - DNA, 49 bp, Artificial Sequence
  Synthetic Construct
gtgatttttgcggactctagagctcttacttgtacaactcgtccattcc

SEQ ID NO: 70 - DNA, 48 bp, Artificial Sequence
  Synthetic Construct
gttctggtggaagtgggggttcatacggcaaggaacagtttttgcgga

SEQ ID NO: 71 - DNA, 48 bp, Artificial Sequence
  Synthetic Construct
ggaggttctggtggaagtgggggttcaatgcatctaccgcccctagag

SEQ ID NO: 72 - DNA, 53 bp, Artificial Sequence
  Synthetic Construct
tgaacccccacttccaccagaacctcccttgtacaactcgtccattcctaaag

SEQ ID NO: 73 - DNA, 22 bp, Artificial Sequence
  Synthetic Construct
ttagcgatcggggaacatgctc

SEQ ID NO: 74 - DNA, 26 bp, Artificial Sequence
  Synthetic Construct
ttagaccttagtggggtgggcgattg

SEQ ID NO: 75 - protein, 15 aa, Artificial Sequence
  Synthetic Construct
KGEFDPAFLYKVVDG

(19) United States
(12) Patent Application Publication
(10) Pub. No.: US 2016/0083738 A1
(43) Pub. Date: Mar. 24, 2016

(54) PRODUCTION OF BACTERIAL MICROCOMPARTMENTS IN EUKARYOTIC CELLS

(71) Applicants: CORNELL UNIVERSITY, Ithaca, NY (US); ROTHAMSTED RESEARCH LTD., Hertfordshire (GB)

(72) Inventors: Maureen HANSON, Ithaca, NY (US); Myat LIN, Ithaca, NY (US); Martin Afan John PARRY, Steppingley, Bedford (GB)

(73) Assignees: CORNELL UNIVERSITY, Ithaca, NY (US); ROTHAMSTED RESEARCH LTD., Hertfordshire (GB)

(21) Appl. No.: 14/889,551

(22) PCT Filed: May 8, 2014
(86) PCT No.: PCT/US14/37398

§ 371 Date: Nov. 6, 2015

Related U.S. Application Data
(60) Provisional application No. 61/820,871, filed on May 8, 2013.
(60) Provisional application No. 61/864,386, filed on Aug. 9, 2013.

(51) Int. Cl. C12N 15/82; C12N 9/88

(52) U.S. Cl. CPC C12N 15/8209; C12N 15/8241; C12N 9/88; C12Y 401/01039

(57) ABSTRACT

The invention relates to eukaryotic organisms such as vascular plants that include recombinant microcompartments.

### CROSS-REFERENCE TO RELATED APPLICATIONS

This application claims benefit of U.S. Provisional Application Nos. 61/820,871 and 61/864,386, filed May 8, 2013 and Aug. 9, 2013, respectively, which are each hereby incorporated by reference in their entirety.

### STATEMENT AS TO FEDERALLY FUNDED RESEARCH

This invention was made with government support under National Science Foundation grant number EF-1105584 and National Institutes of Health Award Number F32GM103019. The government has certain rights in the invention.

### BACKGROUND OF THE INVENTION

The invention, in general, involves expressing microcompartments in eukaryotic cells and targeting proteins to such compartments.

Intracellular compartmentalization is a general strategy used by organisms to carry out metabolic reactions more efficiently. Several bacteria enclose enzymes within proteinaceous polyhedral bodies known as bacterial microcompartments (BMCs) (Bobik Appl. Microbiol. BiotechnoL, 70, 517-725, 2006, Yeates et al. Nature reviews. Microbiology, 6, 681-691, 2008). These microcompartments allow the hosts to overcome unfavorable or challenging metabolic pathways by sequestering volatile or toxic reaction intermediates or concentrating a critical substrate nearby an enzyme that has a slow turnover and low affinity for that substrate. Despite their diverse functions, these microcompartments share a common set of homologous protein subunits, which make up the outer shells in a fashion similar to viral capsids (Yeates et al. Current Opion. Struct. Biol. 21:223-231, 2011).

The β-carboxysome from the freshwater cyanobacterium Synechococcus elongatus PCC7942 is perhaps the best-characterized bacterial microcompartment. It contains two enzymes fundamental to photosynthesis, namely Rubisco (ribulose 1,5-bisphosphate carboxylase/oxygenase) and carbonic anhydrase, and forms an important part of the cyanobacterial CO₂ concentrating mechanism (CCM) (Yeates et al. Nature reviews. Microbiology, 6, 681-691, 2008, Rae et al. Microbiol. Mol. Biol. Rev., 77, 357-379, 2013). While essential to photosynthesis, Rubisco catalyses two competing reactions involving the enediol form of ribulose-1,5-bisphosphate (RuBP or Rubisco). These are the productive carboxylation of RuBP by CO₂ and the wasteful oxygenation of RuBP by molecular oxygen, initiating photorespiration (Long 1991). Carboxysomes increase the concentration of CO₂ around the catalytic site of Rubisco, promoting the carboxylase activity and consequently suppressing the undesired reaction with oxygen (Cannon et al. Appl. Environ. Microbiol., 67, 5351-5361, 2001, Price et al. Journal of experimental botany, 59, 1441-1461, 2008, Whitney et al. Plant Physiol., 155, 27-35, 2011).

### SUMMARY OF THE INVENTION

We have produced synthetic microcompartments in chloroplasts. We also demonstrate that foreign proteins can be targeted to these microcompartments with the use of a signal peptide. Expression of cyanobacterial RuBisco in a C3 plant, tobacco, is also demonstrated.

In general, the invention features a vascular plant including recombinant microcompartments. Exemplary microcompartments are carboxysomes such as alpha-carboxysomes or beta-carboxysomes. In preferred embodiments, the microcompartments are round and slightly elongated. In other preferred embodiments, the microcompartments are about 100-110 nm in length and about 80-90 nm in width; to about 140-160 nm in length and about 110-130 nm in width; to about 50 nm in length and about 300 nm in width; to about 1.5 μm in length and about 1.0 μm in width; or to about 1-3 μm in length and about 1-3 μm in width.

In other preferred embodiments, the microcompartments are located in chloroplasts of the plant. In still other preferred embodiments, the microcompartments are found in the cytoplasm of the plant. Typically, the plants include non-naturally occurring expression constructs (e.g., transgenes) which express at least one microcompartment gene described herein. Such expression constructs are typically stably integrated in the chloroplasts or cytoplasm or both of the plant. In yet other preferred embodiments, the plant is a C3 plant. Exemplary C3 plants include, without limitation, a variety of crop plants such as lettuce, tobacco, petunia, potato, tomato, soybean, carrot, cabbage, poplar, alfalfa, crucifers such as oilseed rape, and sugar beet.

In other preferred embodiments, the microcompartments include a protein substantially identical to CcmO, a protein substantially identical to CcmK2, and a protein substantially identical to CcmL. In yet other preferred embodiments, the microcompartments include a protein substantially identical to CcmO, a protein substantially identical to CcmK2, and a protein substantially identical to CcmM58. In still other preferred embodiments, the microcompartments further include a protein substantially identical to CcmM35 or a protein substantially identical to rbcX or a protein substantially identical to CcmN. And in still other preferred embodiments, the microcompartments further include a protein having substantial identity to a cyanobacterial ribulose bisphosphate carboxylase large subunit or a cyanobacterial ribulose bisphosphate carboxylase small subunit or both. In still another preferred embodiment, the microcompartments further include a protein having substantial identity to a carbonic anhydrase (CcaA).

Preferably, the microcompartment includes a protein having substantial identity to CcmK2, a protein having substantial identity to CcmL, a protein having substantial identity to CcmO, a protein having substantial identity to CcmN, a protein having substantial identity to CcmM58, a protein having substantial identity to CcmM35, a protein having substantial identity to CcaA, a protein having substantial identity to cyanobacterial ribulose bisphosphate carboxylase large subunit, and a protein having substantial identity to cyanobacterial ribulose bisphosphate carboxylase small subunit. And in other preferred embodiments, the microcompartment includes CcmK2, CcmL, CcmO, CcmN, CcmM58, CcmM35, CcaA, a cyanobacterial ribulose bisphosphate carboxylase large subunit, and a cyanobacterial ribulose bisphosphate carboxylase small subunit.

In another aspect, the invention features a method of producing a vascular plant having recombinant microcompartments, the method including expressing in the plant a protein substantially identical to CcmO, a protein substantially identical to CcmK2, and a protein substantially identical to CcmL or substantially identical to CcmM58, wherein expression of the proteins results in production of recombinant microcompartments in the plant. In preferred embodiments, the protein is substantially identical to CcmL.

In other preferred embodiments, the protein is substantially identical to CcmM58. In still other preferred embodiments, the plant further expresses a protein substantially identical to CcmM35 or a protein substantially identical to rbcX or a protein substantially identical to CcmN. And in yet other preferred embodiments, the plant still further expresses a protein substantially identical to a cyanobacterial ribulose bisphosphate carboxylase large subunit or a cyanobacterial ribulose bisphosphate carboxylase small subunit or both. Such plants may also further express a protein substantially identical to carbonic anhydrase (CcaA).

Preferably, the microcompartments found in the plant are round and slightly elongated. The dimensions of such microcompartments are as described herein. As is mentioned above, in preferred embodiments, the microcompartments are located in either chloroplasts or cytoplasm. Preferably, the plant includes non-naturally occurring expression constructs expressing at least one microcompartment gene stably integrated in the chloroplasts of the plant.

In still other preferred embodiments, the plant expresses a protein having substantial identity to CcmK2, a protein having substantial identity to CcmL, a protein having substantial identity to CcmO, a protein having substantial identity to CcmN, a protein having substantial identity to CcmM58, a protein having substantial identity to CcmM35, a protein having substantial identity to CcaA, a protein having substantial identity to cyanobacterial ribulose bisphosphate carboxylase large subunit, and a protein having substantial identity to cyanobacterial ribulose bisphosphate carboxylase small subunit. And preferably, the plant expresses a CcmK2, CcmL, CcmO, CcmN, CcmM58, CcmM35, CcaA, a cyanobacterial ribulose bisphosphate carboxylase large subunit, and a cyanobacterial ribulose bisphosphate carboxylase small subunit.

In another aspect, the invention features a non-human eukaryotic organism including recombinant microcompartments. Exemplary organisms include, without limitation, plants, yeast, non-human mammals, fungi, or insects. The microcompartments produced in the various organisms are as described herein. In preferred embodiments, the microcompartments include a protein substantially identical to CcmO, a protein substantially identical to CcmK2, and a protein substantially identical to CcmL or to CcmM58. In preferred embodiments, the protein is substantially identical to CcmL. In other preferred embodiments, the protein is substantially identical to CcmM58. In preferred embodiments, the microcompartments further include a protein substantially identical to CcmM35 or a protein substantially identical to rbcX or a protein substantially identical to CcmN. And in still other preferred embodiments, the microcompartments further include a protein substantially identical to a cyanobacterial ribulose bisphosphate carboxylase large subunit or to a cyanobacterial ribulose bisphosphate carboxylase small subunit or both. In yet other preferred embodiments, the microcompartments further include a protein substantially identical to a carbonic anhydrase (CcaA). Typically, the organism expresses a protein substantially identical to cyanobacterial rbcL or rbcS or both.

In another aspect, the invention features a cell including recombinant microcompartments. Typical cells for incorporating recombinant microcompartments include bacterial cells, yeast cells, insect cells, plant cells, or mammalian cells. Again, the microcompartments produced in such cells have the characteristics of those described herein.

In still another aspect, the invention features a non-naturally occurring expression cassette including a nucleotide sequence encoding at least one of the following: (i) a protein substantially identical to CcmO; (ii) a protein substantially identical to CcmK2; (iii) a protein substantially identical to CcmL; (ix) a protein substantially identical to CcmN; (v) a protein substantially identical to CcmM58; (vi) a protein substantially identical to CcmM35; (vii) a protein substantially identical to rbcX; (viii) a protein substantially identical to cyanobacterial ribulose bisphosphate carboxylase rbcL; (ix) a protein substantially identical to cyanobacterial ribulose bisphosphate carboxylase rbcS; (x) a protein substantially identical to carbonic anhydrase CcaA; or any combination thereof.

In preferred embodiments, the cassette co-expresses CcmK2 protein, CcmL protein, and CcmO protein or CcmK2 protein, CcmL protein, and CcmM58, or CcmK2 protein, CcmL protein, CcmO protein, and CcmM58 protein. In other preferred embodiments, the cassette is stably integrated into a nuclear genome upon transformation into a host cell. In still preferred embodiments, the cassette is stably integrated into a chloroplast genome upon transformation into a host cell.

In yet other aspect, the invention features a cell including in its genome at least one stably incorporated expression cassette as described herein. In preferred embodiments, the cell is a plant cell in which the expression cassette is stably incorporated in the nuclear genome or chloroplast genome or both of the plant cell.

Cells and organisms described herein are, in general, “transformed” or “transgenic.” These terms accordingly refer to any cell (e.g., a host cell) or organism into which a recombinant or heterologous nucleic acid molecule (e.g., one or more DNA constructs) has been introduced. Thus, the nucleic acid molecule can be stably expressed (e.g., maintained in a functional form in the cell for longer than about three months) or non-stably maintained in a functional form in the cell for less than three months, or in other words is transiently expressed. Transgenic or transformed cells or organisms accordingly contain genetic material not found in untransformed cells or organisms. The term “untransformed” refers to cells that have not been through the transformation process.

The transgenic organisms described herein are generally, but not limited to, transgenic plants or transgenic plant cells, and the recombinant or heterologous nucleic acid molecules (e.g., a transgene) is inserted by artifice into the nuclear or plastidic genomes. Progeny plant or plants deriving from (e.g., by propagating or breeding) the stable integration of heterologous genetic material into a specific location or locations within the nuclear genome or plastidic genome or both of the original transformed cell are generally referred to as a “transgenic line” or a “transgenic plant line.” Transgenic plants or transgenic plant lines thus, for example, contain genetic material not found in an untransformed plant of the same species, variety, or cultivar.

The term “plant” as used herein includes whole plants or plant parts or plant components. By “plant part” or “plant component” is meant a part, segment, or organ obtained from, for example, an intact plant, plant tissue, or plant cell. Exemplary plant parts or plant components include, without limitation, somatic embryos, leaves, seeds, stems, roots, flowers, tendrils, fruits, scions, and rootstocks. Exemplary transformable plants include a variety of vascular plants (e.g., dicotyledonous and monocotyledonous plants as well as gymnosperms) and lower non-vascular plants.

By “plant cell” is meant any self-propagating cell bounded by a semi-permeable membrane and containing a plastid. Such a cell also requires a cell wall if further propagation is desired. Plant cell, as used herein includes, without limitation, algae, microalgae, cyanobacteria, as well as suspension cultures of plant cells such as those obtained from embryos, meristematic regions, callus tissue, leaves, roots, shoots, gametophytes, sporophytes, pollen, and microspores.

As is disclosed herein, the cells and organisms include recombinant microcompartments. Generation of such cells and organisms starts using standard transformation methodologies. The term “transformation” thus generally refers to the transfer of one or more recombinant or heterologous nucleic acid molecule (e.g., a transgene) into a host cell or organism. Methods for introducing nucleic acid molecules into host cells are well known in the art and include, for instance, those methods described herein. By “transgene” is meant any piece of a nucleic acid molecule (e.g., DNA or a recombinant polynucleotide) which is inserted by artifice into a cell, and becomes part of the genome of the organism which develops from that cell. Such a transgene may include a gene which is partly or entirely heterologous (i.e., foreign) to the transgenic organism, or may represent a gene having sequence identity to an endogenous gene of the organism.

Recombinant microcompartments are typically generated utilizing recombinant polynucleotides which, in turn, are transcribed and translated resulting in the production of recombinant polypeptides. A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid. A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Alternatively, or additionally, the isolated polypeptide is separated from other cellular components with which it is typically associated, e.g., by any of the various protein purification methods known in the art.

By “polypeptide” or “protein” is meant any chain of amino acids, regardless of length or post-translational modification (for example, glycosylation or phosphorylation).

Described herein are various polynucleotides and polypeptides useful in producing microcompartments and carboxysomes including ccmP, CcmP, ccmO, CcmO, ccmK2, CcmK2, ccmL, CcmL, ccmM35, CcmM35, ccmM58, CcmM58, Synechococcus LS (Rubisco large subunit) nucleotide sequence, Synechococcus LS (Rubisco large subunit), Synechococcus SS (Rubisco small subunit) nucleotide sequence, Synechococcus SS (Rubisco small subunit), rbcX, RbcX, ccmM35, CcmM35, ccmK3, CcmK3, ccmK4, CcmK4, ccaA, CcaA (carbonic anhydrase), ccmN, and CcmN (FIG. 25).

It is understood that polynucleotides and polypeptides having substantial identity to such molecules are also useful in the methods disclosed herein. By “having substantial identity to” or by “substantially identical to” is meant a polynucleotide or polypeptide exhibiting at least 50%, preferably 70%, 75%, 85%, or 85%, more preferably 90%, and most preferably 95%, 96%, 97%, 98%, and 99% homology (or identity) to a reference nucleic acid or sequence. For nucleic acids, the length of comparison sequences will generally be at least 50 nucleotides, preferably at least 60 nucleotides, more preferably at least 75 nucleotides, and most preferably 110 nucleotides. For polypeptides, the length of comparison sequences will generally be at least 16 amino acids, preferably at least 20 amino acids, more preferably at least 25 amino acids, and most preferably 35 amino acids. Sequence identity is typically measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various substitutions, deletions, substitutions, and other modifications. Conservative substitutions typically include substitutions within the following groups: glycine alanine; valine, isoleucine, leucine; aspartic acid, glutamic acid, asparagine, glutamine; serine, threonine; lysine, arginine; and phenylalanine, tyrosine.

In view of the results disclosed herein, the invention, in general, also features a non-human eukaryotic organism that includes a carboxysome (e.g., a bacterial carboxysome). In preferred embodiments, the carboxysome is a cyanobacterial carboxysome such as Synechococcus elongatus strain 7942 β carboxysome. In other preferred embodiments, the carboxysome includes Synechococcus elongatus strain 7942 (a) CcmK2, CcmO, and CcmL proteins or (b) CcmK2, CcmO, and CcmM58 proteins. Exemplary microcompartments are located in a eukaryotic organelle such as a chloroplast. Alternatively, the microcompartments are located in a cell's cystoplasm.

In another aspect, the invention features a cell including a heterologous microcompartment. In preferred embodiments, the heterologous microcompartment includes Synechococcus elongatus strain 7942 β carboxysomal proteins. In other preferred embodiments, the heterologous microcompartment includes Synechococcus elongatus strain 7942 proteins such as (a) CcmK2 and CcmO proteins or (b) CcmK2, CcmO, and CcmL or CcmM58 proteins. Exemplary heterologous microcompartments may be located in a eukaryotic organelle such as a chloroplast. Alternatively, the heterologous microcompartment may be located in a cell's cystoplasm.

In another aspect, the invention features an expression cassette that includes a bacterial microcompartmental gene isolated from a bacterium, wherein the expression cassette expresses CcmK2 protein, CcmO protein, and CcmL protein or CcmM58 or combinations thereof. In preferred embodiments, the expression cassette co-expresses CcmK2 protein, CcmL protein (or CcmM58 protein), CcmO protein. In other preferred embodiments, expression cassette co-expresses CcmK2 protein and CcmO protein. In another preferred embodiment, the expression cassette expresses CcmK2 protein. In still another preferred embodiment, the expression cassette expresses CcmL protein. And still another preferred embodiment, the expression cassette expresses CcmO protein. Sequences encoding such proteins or any bacterial microcompartmental gene may be employed according to any preferred codon optimization known in the art for increasing expression in a transformed organism.

In yet another aspect, the invention features an isolated polypeptide including a sequence having the amino acid sequence

A-X-B,
wherein A is a polypeptide;
wherein X is a linker peptide; and
wherein B is YGKEQFLRMRQSMFPDR.
   By “linker” is meant an amino acid sequence of one or more amino acids in length, e.g., that is not cleavable, for example, by auto-cleavage, enzymatic, or chemical cleavage. The linker can include nonpolar, polar, and/or charged amino acids. In some embodiments, linkers include or consist of flexible portions, e.g., regions without significant fixed secondary or tertiary structure. Exemplary flexible linkers are glycine-rich linkers, e.g., containing at least 50%, 60%, 65%, 70%, 75%, 80%, 85%, 90%, 95%, or even 100% glycine residues. Linkers may also contain, e.g., serine residues. In some cases, the amino acid sequence of linkers consists only of glycine and serine residues. A linker can be, for example, 1 to 30 amino acids in length, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. In preferred embodiments, the linker is GGSGGSGGS.

In still another aspect, the invention features an expression cassette including a gene expressing the isolated polypeptide including a sequence having the amino acid sequence

A-X-B,
wherein A is a polypeptide;
wherein X is a linker peptide; and
wherein B is YGKEQFLRMRQSMFPDR.
   In preferred embodiments, the linker is GGSGGSGGS.

In still another aspect, the invention features a cell including an expression cassette that expressed the A-X-B polypeptide.

Other features and advantages of the invention will be apparent from the following Detailed Description and the claims.

### BRIEF DESCRIPTION OF THE DRAWINGS

FIG. 1 shows a carboxysome of Synechococcus elongatus PCC 7942.

FIG. 2 shows 16 constructs for expressing carboxysomal genes. LB refers to left border (a standard feature necessary for agroinfiltration); RB refers to right border (a standard feature necessary for agroinfiltration); bar refers to bialaphos resistance gene for selection (a commonly used marker in plants); Pnos refers to nos promoter; ‘pAnos refers to nos terminator; P35SS refers to 35SS promoter; ‘pA35S refers to 35S terminator; attB1, attB2 refers to gateway cloning sites; recActp refers to DNA sequence encoding the chloroplast transit peptide from Arabidopsis thaliana recA gene (ATGGATTCACAGCTAGTCTTGTCTCTGAAGCTGAATCCAAGCTTCACTCCTCTTTCTCCTCTCTTCCC TTTCACTCCATGTTCTTCTTTTTCGCCGTCGCTCCGGTTTTCTTCTTGCTACTCCCGCCGCCTCTATTC TCCGGTTACCGTCTACGCCGCGAAGAAACTCTCCCACAAAATCAGTTCTGAATTCGAT); ccmK2 refers to ccmK2 gene from cyanobacterium Synechococcus elongatus PCC7942 encoding the CcmK2 protein (“ccm” stands for “CO₂ concentration mechanism”); ccmL refers to ccmL gene from cyanobacterium Synechococcus elongatus PCC7942 encoding the CcmL protein; ccmO refers to ccmO gene from cyanobacterium Synechococcus elongatus PCC7942 encoding the CcmO protein; ccmM58 refers to ccmM58 gene from cyanobacterium Synechococcus elongatus PCC7942 encoding the CcmM58 protein; Puq10 refers to ubiquitin 10 promoter from Arabidopsis; ccfp refers to the gene encoding ceruleum fluorescent protein CCFP; Pmas refers to MAS promoter; N17 refers to the DNA sequence encoding the last 17 amino acids of the CcmN protein (TACGGCAAGGAACAGTTTTTGCGGATGCGCCAGAGCATGTTCCCCGATCGC); and L9 refers to the DNA sequence encoding the linker peptide between YFP and N17 (GGAGGTTCTGGTGGAAGTGGGGGTTCA)

FIG. 3 shows the results of transient expression of β-carboxysome proteins expressed from construct #1, construct #2, and construct #3.

FIG. 4 shows the results of transient expression of β-carboxysome proteins expressed from construct #4 and construct #5.

FIG. 5 shows the results of transient expression of β-carboxysome proteins expressed from construct #6 and construct #7.

FIG. 6 shows the results of transient expression of β-carboxysome proteins expressed from construct #8, construct #9, construct #10, and construct #11.

FIG. 7 shows the results of transient expression of β-carboxysome proteins expressed from construct #12.

FIG. 8 shows the results of transient expression of β-carboxysome proteins expressed from construct #13 and construct #14.

FIG. 9 shows the first gene (ccmK2 shown in the example) is inserted by Gateway cloning and driven by P35SS promoter; the second and third operons are inserted at the AscI and MluI sites respectively; recActp is used in all proteins if desirable to target them into chloroplasts.

FIG. 10A shows that β-carboxysomal proteins are transiently expressed in tobacco and N. benthamiana leaves following agroinfiltration. Each expression vector contains 1-3 transgenes driven by different promoters.

FIG. 10B shows that when expressed individually, CcmK2 and CcmL result in abnormal rod shapes. CcmO signal is diffuse and when they are simultaneously expressed, punctate fluorescent signals are seen.

FIG. 11 shows confocal image of N. benthamiana leaf cells transiently expressing stroma-targeted YFP-tagged β-carboxysomal shell proteins. (a) CcmK2-YFP by 35SS promoter; (b, c) CcmO-YFP by 35SS promoter; (d) CcmO-YFP by 35SS promoter and CcmL by ubiquitin-10 promoter. (e) CcmO-YFP by 35SS promoter and CcmK2 by ubiquitin-10 promoter; and (f) CcmO-YFP by 35SS promoter, CcmK2 by ubiquitin-10 promoter and CcmL by ubiquitin-10 promoter. Green=YFP fluorescence. Red=chlorophyll autofluorescence. Line bars (a-f)=5 μm.

FIG. 12 shows ultrathin sections of leaf mesophyll cells from agroinfiltrated N. benthamiana expressing the indicated carboxysomal proteins: (a,d) CcmO-YFP; (b,e) CcmO-YFP and CcmK2; (c,f-i) CcmO-YFP, CcmK2 and CcmL-YFP. Images showing different structures within the chloroplast stroma: (a,d, black arrows) protein aggregates; (b,e, black arrows) protein aggregates organized in parallel line structures; (c,f-i, black arrows) circular structures resembling a carboxysome shell. (a-c) Leaf tissue prepared by normal chemical fixation; and (d-i) leaf tissue prepared by high pressure freeze fixation (HPF) in combination with immunogold labelling: (d-g) GFP labelling; (h) CcmK2 labelling; (i) CcmO labelling. A secondary antibody conjugated with 10 nm gold particles was used for imaging. (a-e) line bar=500 nm; (f) line bar=1 μm; (g-i) line bar=200 nm; (detail panel c) line bar=50 nm; (detail panel e) line bar=100 nm; (detail panel f) line bar=200 nm.

FIG. 13 shows (a) schematic representation of a circular structure. (b) High magnification of a circular structure in the chloroplast stroma of a mesophyll cell from agroinfiltrated N. benthamiana expressing CcmO-YFP, CcmK2 and CcmL-YFP. Leaf tissue prepared by normal chemical fixation. Line bar=50 nm.

FIG. 14 shows ultrathin sections of leaf mesophyll cells from agroinfiltrated N. benthamiana expressing CcmO-YFP, CcmK2 and CcmL-YFP. Images showing different structures into the chloroplast stroma: (a,d, black arrows) elongated structures; (b,e, black arrows) disorganized structures; (c,f, black arrows) intermediate structures; (a-c) Leaf tissue prepared by normal chemical fixation; and (d-f) Leaf tissue prepared by high pressure freeze fixation (HPF) in combination with immunogold labelling. An antibody against GFP and a secondary antibody conjugated with 10 nm gold particles were used. (a,d) Line bar=1 μm; (b,c,f) line bar=200 nm; (e) line bar=500 nm; (detail panel a,d) line bar=200 nm.

FIG. 15 shows ultrathin sections of leaf mesophyll cells from agroinfiltrated N. benthamiana expressing the indicated carboxysomal proteins: (a-g) CcmO-YFP, CcmK2 and CcmM58-YFP; (h,i) CcmO-YFP, CcmK2, CcmL and CcmM58-YFP; (a-e,h, black arrows) Images showing circular structures resembling a carboxysome shell; and (f,g,i, black arrows) images showing elongated structures (parallel lines spaced 8-9 nm and organized forming a net structure are indicated). Leaf tissue prepared by high pressure freeze fixation without immunogold labelling (a,f), and in combination with immunogold labelling (b-e, g-i). Different antibodies against carboxysomal proteins were used: (b,g-i) GFP labelling; (c) CcmK2 labelling; (d) CcmO labelling; (e) CcmM58 labelling. A secondary antibody conjugated with 10 nm gold particles was reacted with the primary antibodies. (a) Line bar=100 nm; (b-e,g-i) line bar 200 nm; (f) Line bar=50 nm.

FIG. 16 shows confocal image of N. benthamiana leaf cells transiently expressing stroma-targeted CcmK2, CcmO and CcmN to determine co-localization. (a) CcmN-CFP by 35SS promoter. CFP fluorescence is shown in cyan and chlorophyll autofluorescence in red. (b-d) CcmN-CFP by 35SS promoter, CcmK2 by ubiquitin-10 promoter and CcmO-YFP by MAS promoter. Individual CFP (b, in cyan) and YFP (c, in yellow) channels as well as the merged channel (d, CFP in cyan and YFP in yellow) from the same region are shown. (e,f) CcmNd17-CFP by 35SS promoter, CcmK2 by ubiquitin-10 promoter and CcmO-YFP by MAS promoter. Individual CFP (e, in cyan) and YFP (f, in green) channels of the same leaf section are shown. (g,h) CcmO-CFP by 35SS promoter, CcmK2 by MAS promoter and YFP-CcmN17 by ubiquitin-10 promoter. Individual CFP (g, in cyan) and YFP (h, in green) channels of the same leaf section are shown. Line bars (a-h)=5 μm.

FIG. 17 shows ultrathin sections of leaf mesophyll cells from agroinfiltrated N. benthamiana expressing the indicated carboxysomal proteins: (a-c) CcmO-YFP, CcmK2 and CcmM58-YFP; (d-f) CcmO-YFP, CcmK2, CcmL and CcmM58-YFP. (a) Images showing a chloroplast with circular structures (asterisk) and a chloroplast with elongated structures (black arrow). (b-f, black arrows) Images showing elongated structures in the chloroplast stroma. Leaf tissue prepared by high pressure freeze fixation in combination with immunogold labelling. Different antibodies against carboxysomal proteins were used: (b,d) CcmK2 labelling; (c,e) CcmO labelling; (f) CcmM58 labelling. A secondary antibody conjugated with 10 nm gold particles was used for imaging. (a) Line bar=1 μm; (b) line bar=500 nm; (c-f) line bar 200 nm.

FIG. 18 shows confocal image of N. benthamiana leaf cells transiently expressing stroma-targeted CcmN17-CFP by 35SS promoter, CcmK2 by MAS promoter and CcmO-YFP by ubiquitin-10 promoter to determine co-localization. Individual channels of chlorophyll autofluorescence (a, in red), CFP (b, in cyan) and YFP (c, in green) from the same leaf section are shown. The merged image (d) of all three channels is shown in the same colors. Line bars (a-d)=5 μm.

FIG. 19 shows ultrathin sections of leaf mesophyll cells from agroinfiltrated N. benthamiana expressing the indicated carboxysomal proteins: (a,b) CcmN-CFP, CcmK2 and CcmO-YFP; (c,d) YFP-CcmN17, CcmK2 and CcmO-YFP. Leaf tissue prepared by high pressure freeze fixation in combination with immunogold labelling. Different primary antibodies were used: (a,c,d) GFP labelling; (b) CcmN labeling. A secondary antibody conjugated with 10 nm gold particles was used for imaging. Black arrows indicate specific labelling into chloroplast stroma. (a) Line bar=500 nm; (b, d) line bar=200 nm; (c) line bar 400 nm; (detail panel a,c) Line bar=200 nm.

FIG. 20 shows the schematic of agrobacterial expression vectors. (a) An example vector with one carboxysomal gene cloned between the Gateway cassettes under the 35SS promoter. (b) An example vector with two additional operons driven by ubiquitin-10 and MAS promoters inserted at the AscI site to co-express three carboxysomal genes.

FIG. 21 shows a tobacco chloroplast transformant harboring ccmK2-ccmL-T3-ccmM58-T1-IEE-ccmO-yfp-T2-PpsbA-ccmN-T4-IEE-aadA-T5 and the resulting microcompartments found in a chloroplast. IEE refers to intercistronic expression element; T1-8 refers to terminators; PpsbA refers to a promoter from tobacco chloroplast psbA gene; and aadA refers to spectinomycin resistance selectable marker gene. After infiltration, leaves were examined by electron immunomicroscopy.

FIG. 22 shows a tobacco chloroplast transformant harboring ccmK2-T7-IEE-ccmM58-T6-IEE-ccaA-T8-IEE-ccmO-yfp-ccmO-yfp-ccmL-PpsbA-GfpDB-ccmM35-T4-IEE-ccmN-T1-IEE-aadA-T5 and the resulting microcompartments found in a chloroplast. GfpDB refers to the N-terminal 14 amino-acid long peptide of gfp gene fused as a downstream box. After infiltration, leaves were examined by electron immunomicroscopy.

FIG. 23 shows the chloroplast transformants with tobacco rbcL replaced by cyanobacterial rbcL, rbcS and beta-carboxysomal genes and a resulting microcompartment found in a chloroplast.

FIG. 24 shows a tobacco chloroplast transformant exhibiting an active cyanobacterial Rubisco.

FIG. 25 shows the nucleotide and amino acid sequences for ccmP, CcmP, ccmO, CcmO, ccmK2, CcmK2, ccmL, CcmL, ccmM35, CcmM35, ccmM58, CcmM58, Synechococcus LS (Rubisco large subunit) nucleotide sequence, Synechococcus LS (Rubisco large subunit), Synechococcus SS (Rubisco small subunit) nucleotide sequence, Synechococcus SS (Rubisco small subunit), rbcX, RbcX, ccmM35, CcmM35, ccmK3, CcmK3, ccmK4, CcmK4, ccaA, CcaA (carbonic anhydrase), ccmN, and CcmN.

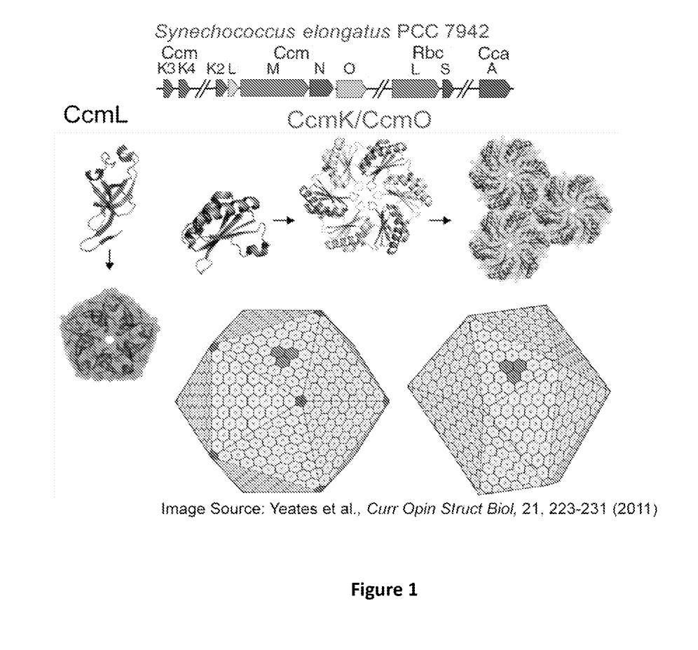

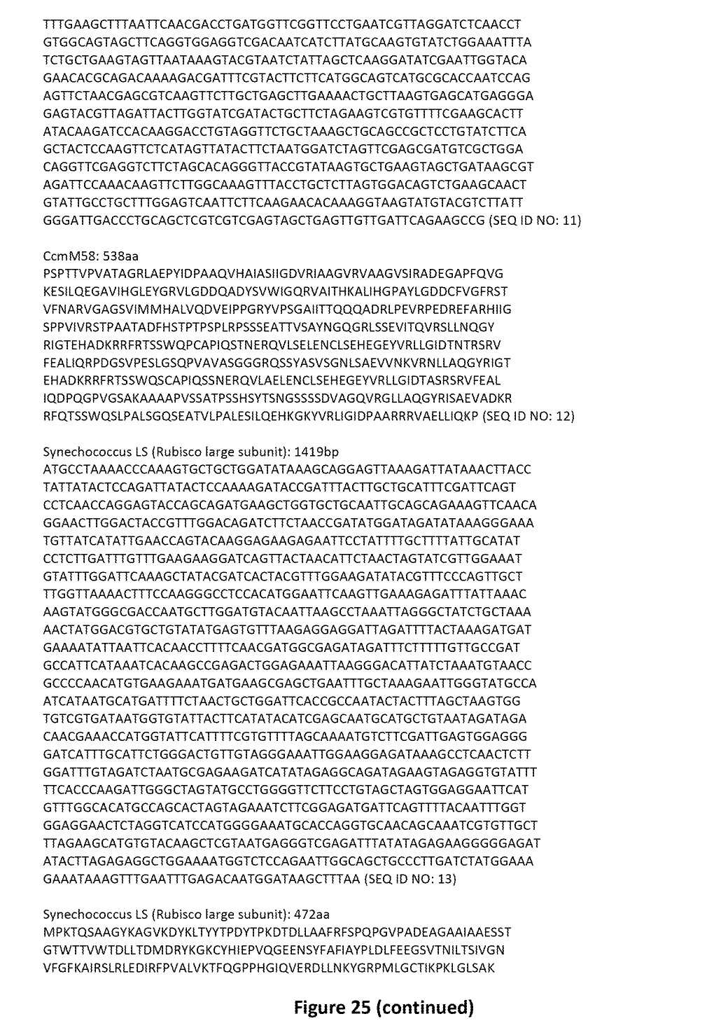

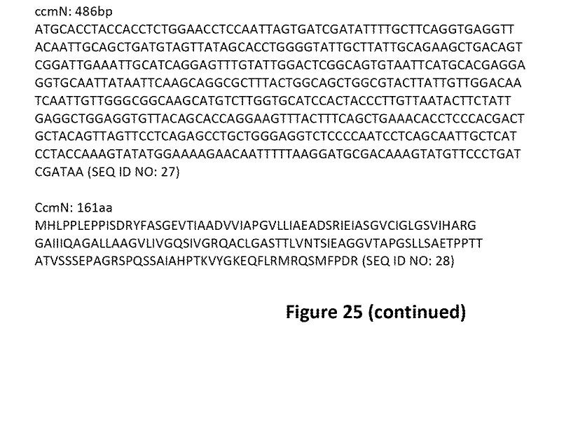

The patent or application file contains drawings (FIGS. 1-24) executed in color. Copies of this patent or patent application publication with color drawings will be provided by the Office upon request and payment of the necessary fee.

### DETAILED DESCRIPTION OF THE INVENTION

Cyanobacteria have evolved a carbon dioxide-concentrating mechanism (CCM) to overcome the poor kinetic properties of ribulose bisphosphate carboxylase oxygenase (Rubisco), a key carbon-fixing enzyme in the Calvin cycle. The main features of the cyanobacterial CCM include (i) active uptake of inorganic carbon species such as carbon dioxide and bicarbonate ion, and (ii) carboxysome microcompartments where carbon dioxide is accumulated at the site of Rubisco. As is described herein, we target multiple proteins such as CcmK2, CcmO, and CcmL or CcmM58 into chloroplasts. First, by agroinfiltration we performed transient expression of these foreign proteins fused with transit sequences and fluorescent tags to investigate the co-localization of these proteins in chloroplasts. Then we generated stable tobacco chloroplast transformants in which multiple carboxysomal proteins are simultaneously expressed. Microscopy is used to characterize the structures formed in the chloroplasts. By properly expressing all necessary carboxysome structural components, assembling of β-carboxysome microcompartments in plant chloroplasts has been achieved.

We have also expressed multiple carboxysomal proteins in plant cells (either tobacco or the related species Nicotiana benthamiana). Synechococcus CcmK2 is one of the proteins with Pfam00936 domain or BMC (bacterial microcompartment) domain, Synechococcus CcmO is a protein with tandem BMC domain and Synechococcus CcmL is a protein with Pfam03319 domain. Expression of the Synechococcus CcmK2, CcmO and CcmL or CcmM58 proteins in chloroplasts of tobacco results in the production of microcompartment shells.

When CcmK2, CcmL, and CcmO are individually targeted to chloroplasts, no microcompartments are formed. The co-expression of CcmK2 and CcmO results in the assembly of empty microcompartments. The co-expression of CcmK2, CcmL and CcmO also results in the assembly of empty microcompartments.

Using standard methods, the proteins can be expressed stably from the nuclear genome or the chloroplast genome, and can be targeted to the chloroplast or to other subcellular localizations using appropriate targeting sequences. We have, for example, targeted the shell proteins to chloroplasts, but they could also be expressed and assembled in the cytoplasm.

We have co-localized foreign proteins with β-carboxysomes by expressing them with specific signal peptides. One such signal peptide is a 17 amino acid sequence that is placed at the C terminus of the protein to be co-localized. A linker between the signal peptide and the protein to be co-localized is also introduced. Yellow Florescence Protein (YFP) is used to demonstrate co-localization of proteins to microcompartments. This means that proteins can be deliberately incorporated into microcompartments by the use of this targeting signal.

The sequence of the 17 amino acid peptide is YGKEQFLRMRQSMFPDR, from the C-terminus of the Synechococcus CcmN protein.

In particular, we have also designed a linker between the protein to be co-localized and the signal peptide in order to minimize any unwanted interaction between the signal peptide and the protein of interest. The amino acid sequence of the linker we used is GGSGGSGGS.

Carboxysomes may be engineered into virtually any eukaryotic cell according to standard methods known in the art such as mammalian, yeast, and insect cells. Engineering of plants and plant cells to include microcompartments and carboxysomes is especially preferred and is described as follows.

Plant Expression Constructs

The construction of nuclear expression cassettes for use in virtually any plant, such as in C3 plants, is well established. Expression cassettes are DNA constructs where various promoter, coding, and polyadenylation sequences are operably linked. In general, expression cassettes typically include a promoter that is operably linked to a sequence of interest which is operably linked to a polyadenylation or terminator region. In certain instances including, but not limited to, the expression of transgenes in a plant, it may also be useful to include an intron sequence to enhance expression. A variety of promoters can be used as well. One broad class of useful promoters is referred to as “constitutive” promoters in that they are active in most plant organs throughout plant development. For example, the promoter can be a viral promoter such as a CaMV35S promoter. The CaMV35S promoters are active in a variety of transformed plant tissues and most plant organs (e.g., callus, leaf, seed and root). Enhanced or duplicate versions of the CaMV35S promoters are particularly useful as well. Other useful promoters are known in the art.

Promoters that are active in certain plant tissues (i.e., tissue specific promoters) can also be used to drive expression of a carboxysome proteins disclosed herein. Transcriptional enhancer elements can also be used in conjunction with any promoter that is active in a plant cell or with any basal promoter element that requires an enhancer for activity in a plant cell. Transcriptional enhancer elements can activate transcription in various plant cells and are usually 100-200 base pairs long. The enhancer elements can be obtained by chemical synthesis or by isolation from regulatory elements that include such elements, and can comprise additional flanking nucleotides that contain useful restriction enzyme sites to facilitate subsequence manipulation. Enhancer elements can be typically placed within the region 5′ to the mRNA cap site associated with a promoter, but can also be located in regions that are 3′ to the cap site (i.e., within a 5′ untranslated region, an intron, or 3′ to a polyadenylation site) to provide for increased levels of expression of operably linked genes. Such enhancers are well known in the art. A polyadenylation signal provides for the addition of a polyadenylate sequence to the 3′ end of the RNA. The Agrobacterium tumor-inducing (Ti) plasmid nopaline synthase (NOS) gene 3′ and the pea ssRUBISCO E9 gene 3′ untranslated regions contain polyadenylate signals and represent non-limiting examples of such 3′ untranslated regions that can be used in constructing an expression cassette. It is understood that this group of exemplary polyadenylation regions is non-limiting and that one skilled in the art could employ other polyadenylation regions that are not explicitly cited here.

Additionally 5′ untranslated leader sequences can be operably linked to a coding sequence of interest in a plant expression cassette. Thus the plant expression cassette can contain one or more 5′ non-translated leader sequences which serve to increase expression of operably linked nucleic acid coding sequences encoding any of the polypeptides described herein.

Sequences encoding peptides that provide for the localization of any of the polypeptides described herein in to plastids can be operably linked to the sequences that encode the particular polypeptide. Transit sequences for incorporating nuclear-encoded proteins into plastids are well known in the art.

It is anticipated that any of the aforementioned plant expression elements can be used with a polynucleotide designed so that they will express one or more of the polypeptides encoded by any of the polynucleotides described herein in a plant or a plant part. Plant expression cassettes including one or more of the polynucleotides described herein which encode one or more of their respective polypeptides, that will provide for expression of one or more polypeptides in a plant are provided herein.

The DNA constructs that include the plant expression cassettes described above are typically maintained in various vectors. Vectors contain sequences that provide for the replication of the vector and covalently linked sequences in a host cell. For example, bacterial vectors will contain origins of replication that permit replication of the vector in one or more bacterial hosts. Agrobacterium-mediated plant transformation vectors typically comprise sequences that permit replication in both E. coli and Agrobacterium as well as one or more “border” sequences positioned so as to permit integration of the expression cassette into the plant chromosome. Selectable markers encoding genes that confer resistance to antibiotics are also typically included in the vectors to provide for their maintenance in bacterial hosts.

Much of the discussion above, which concerns nuclear transformation, is relevant to introduction of genes into plastids and chloroplasts, but there are some differences (see, for example, Hanson et al., Journal of experimental botany 64: 731-742, 2013). For example, polyadenylation signals are not placed on chloroplast transgenes; instead plastid 3′ stability sequences must be incorporated. Unlike typical Agrobacterium-mediated nuclear transformation, there is a simple method to ensure proper targeting of a transgene to a location of interest within the plastid genome, by surrounding the transgene with plastid DNA sequences so that homologous recombination will occur. Selection of proper promoter and 5′ untranslated region sequences are important, and sometimes a suitable “downstream box” at the beginning of the translated region is needed to modulate expression (see, for example, Gray et al., Biotechnology and bioengineering 102: 1045-1054, 2009). Furthermore, because plastid genes can be transcribed in operons, to optimize expression an intercistronic expression element (IEE) can be used so that monocistronic transcripts are obtained for better expression levels (see, for example, Zhou et al., The Plant journal: for cell and molecular biology 52: 961-972, 2007). No plastid transit sequence is needed on the plastid transgene since expression occurs from within the plastid.

Transgenic Plants and Methods for Transgenic Plants Including Carboxysome Proteins

Methods of obtaining a transgenic plant (or a transgenic plant part) including a recombinant microcompartment are also provided by this invention. First, expression vectors suitable for expression of any of the polypeptides disclosed herein plants are introduced into a plant, a plant cell or a plant tissue using transformation techniques according to standard methods well known in the art. Next a transgenic plant containing the plant expression vector is obtained by regenerating that transgenic plant from the plant, plant cell or plant tissue that received the expression vector. The final step is to obtain a transgenic plant that expresses a carboxysome protein and, preferably, a microcompartment.

Plant expression vectors can be introduced into the chromosomes of a host plant via methods such as Agrobacterium-mediated transformation, particle-mediated transformation, DNA transfection, or DNA electroporation, or by so-called whiskers-mediated transformation. Exemplary methods of introducing transgenes are well known to those skilled in the art.

Those skilled in the art will further appreciate that any of these gene transfer techniques can be used to introduce the expression vector into the chromosome of a plant cell, a plant tissue, a plant, or a plant part.

When the plant expression vector is introduced into a plant cell or plant tissue, the transformed cells or tissues are typically regenerated into whole plants by culturing these cells or tissues under conditions that promote the formation of a whole plant (i.e., the process of regenerating leaves, stems, roots, and, in certain plants, reproductive tissues). The development or regeneration of transgenic plants from either single plant protoplasts or various explants is well known in the art. This regeneration and growth process typically includes the steps of selection of transformed cells and culturing selected cells under conditions that will yield rooted plantlets. The resulting transgenic rooted shoots are thereafter planted in an appropriate plant growth medium such as soil. Transgenic plants having incorporated into their genome transgenic DNA segments encoding one or more of the polypeptides described herein are within the scope of the invention. It is further recognized that transgenic plants containing the DNA constructs described herein, and materials derived therefrom, may be identified through use of PCR or other methods that can specifically detect the sequences in the DNA constructs.

Once a transgenic plant is regenerated or recovered, a variety of methods can be used to identify or obtain a transgenic plant that includes one or more of the polypeptides described herein as well as includes a carboxysome. One general set of methods is to perform assays that measure the amount of the polypeptide that is produced. Alternatively, the amount of mRNA produced by the transgenic plant can be determined to identify plants that express of the polypeptide. Standard microscopic methods are also useful to identify plants engineered to include carboxysomes.

Below we describe a transient expression method to explore the possibility of transferring components of β-carboxysomes from Synechococcus PCC7942 into plant chloroplasts. Agroinfiltration of Nicotiana benthamiana leaves gave rise to high levels of protein expression, demonstrating that carboxysomal proteins can be produced in plant cells and correctly targeted into the chloroplast stroma when fused to a chloroplast transient peptide. Plastid transgenes were also introduced by particle bombardment into the Nicotiana tabacum plastid genome. Application of both fluorescence and transmission electron microscopy in combination with immunogold labelling enabled visualization of assemblies of carboxysomal proteins in the chloroplast stroma. We also describe the production of transformants in which the transgenes of interest are incorporated into the chloroplast genome. Also demonstrated is expression of a cyanobacterial Rubisco expressed in transgenic tobacco.

### EXAMPLES

The following Examples are intended to illustrate, not limit, the invention.

### Example 1

### Synechococcus elongatus PCC7942 β Carboxysomes

The Examples described herein are directed to synthesis of Synechococcus elongatus PCC7942 β microcompartments and carboxysomes. Referring to FIG. 1, carboxysome shells are made up of proteins.

### Example 2

### Introduction of Operons into Chloroplast DNA

Plastid expression operons containing different combinations of β-carboxysomal genes may be introduced into various locations in a chloroplast genome such as accD-rbcL-atpB, trnfM-trnG, trnV-rps12, and rrn16-trnI-trna of the tobacco chloroplast DNA.

### Example 3

### Constructs Used to Express a Single Microcompartment Gene

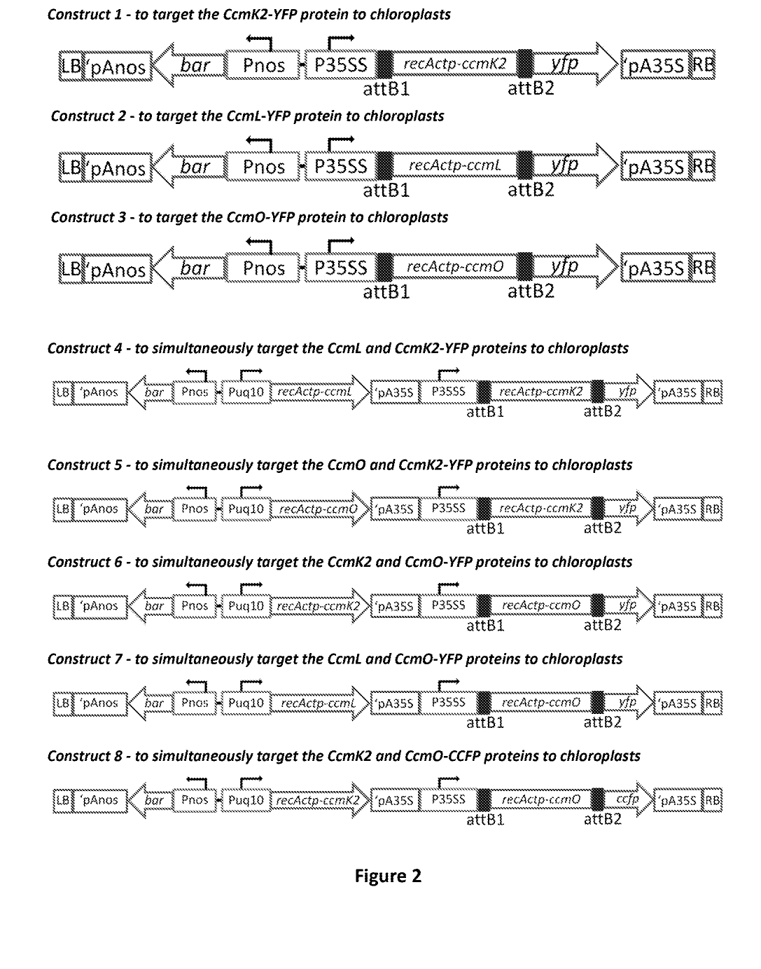

Constructs useful in the invention are depicted in FIG. 2. Such constructs are engineered according to standard methods known in the art.

### Example 4

### Transient Expression of 6-Carboxysome Proteins in Chloroplasts

Expression vectors described in Example 3 were transiently expressed in a transient expression system (Sparkes et al. Nat. Protocols 1: 2019-2025, 2006) using Agrobacterium tumefaciens (GV3101/pMP90RK) according to standard methodologies. The leaves of Nicotiana benthamiana were infiltrated with agrobacterial culture. Approximately 2 to 3 days after agroinfiltration, the proteins transiently expressed in leaf cells were monitored using laser-scanning confocal microscopy.

### Example 5

### CcmK2-YFP (Construct #1), CcmL-YFP (Construct #2), and CcmO-YFP (Construct #3)

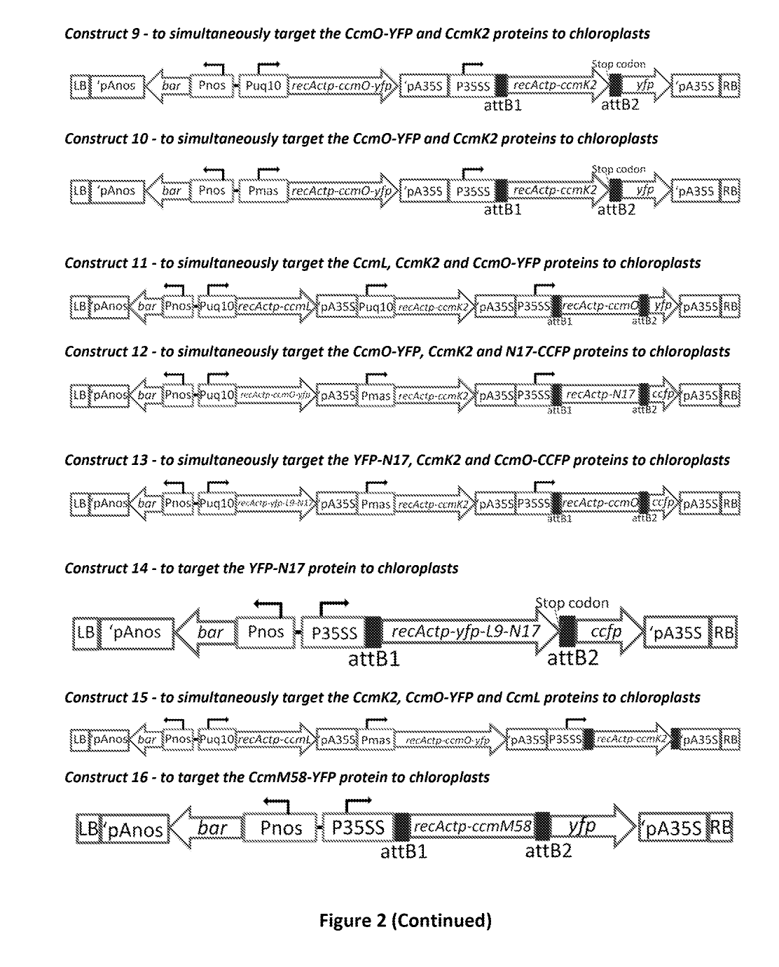

CcmK2-YFP (expressed from construct #1) and CcmL-YFP (construct #2) were found to self polymerize into elongated structures. CcmO-YFP (construct #3) gives diffuse fluorescent signal. These results are shown in FIG. 3.

### Example 6

### CcmK2-YFP (P35SS) CcmL (Pug10) (Construct #4) and CcmK2-YFP (P35SS) CcmO (Pug10) (Construct #5)

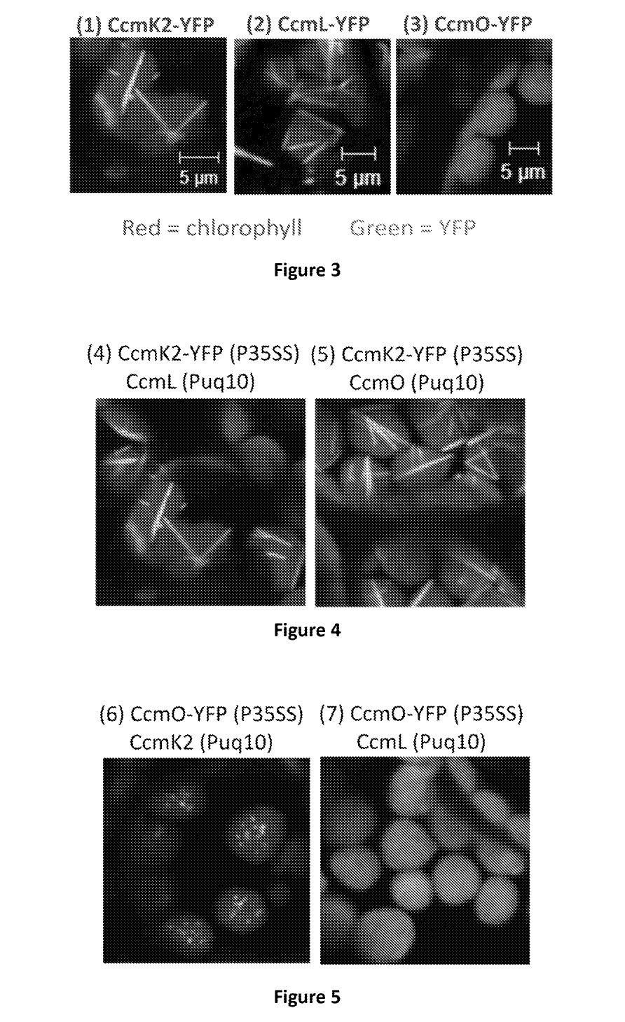

Co-expression of CcmK2-YFP and CcmL (construct #4) gives rise to elongated structures similar to those formed when only CcmK2-YFP is expressed (construct #1). Co-expression of CcmK2-YFP and CcmO (construct #5) results in similar elongated structures. These results are shown in FIG. 4.

### Example 7

### CcmO-YFP (P35SS) CcmK2 (Pug10) (Construct #6) and CcmO-YFP (P35SS) CcmL (Pug10) (Construct #7)

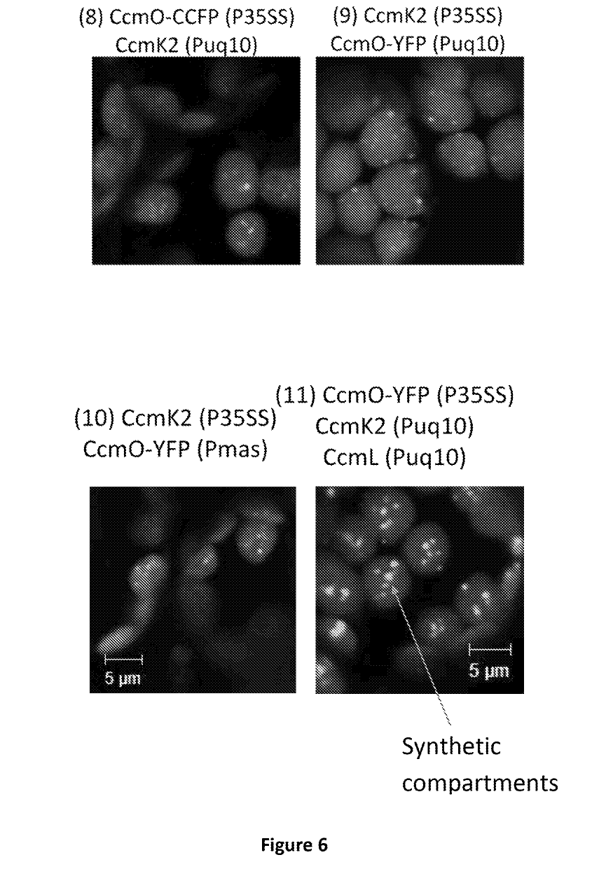

Co-expression of CcmO-YFP and CcmK2 (construct #6) gives rise punctate loci yielding a fluorescent signal but it is unlikely that microcompartments were formed. Co-expression of CcmO-YFP and CcmL (construct #7) does not form any structure and only diffuse fluorescent signal is observed. These results are shown in FIG. 5.

### Example 8

### CcmO-CCFP (P35SS) CcmK2 (Pug10) (Construct #8), CcmK2 (P35SS) CcmO-YFP (Pug10) (Construct #9), CcmK2 (P35SS) CcmO-YFP (Pmas) (Construct #10), and CcmO-YFP (P35SS) CcmK2 (Pug10) CcmL (Pug10) (Construct #11)

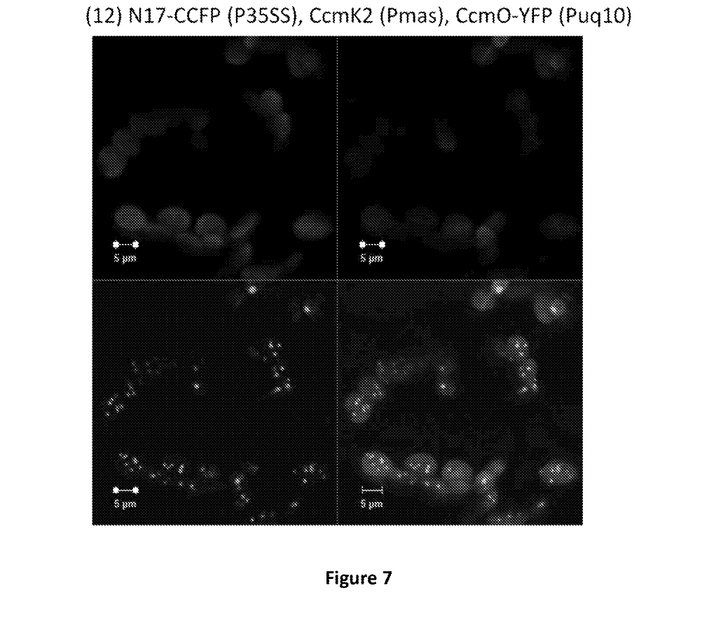

Co-expression of CcmO-CCFP and CcmK2 (construct #8) gives rise punctate loci associated with the formation of empty microcompartments. Microcompartments are also formed when CcmK2 and CcmO-YFP are co-expressed (construct #9 and #10). Note that constructs #6, #9 and #10 use different combinations of promoters to target CcmK2 and CcmO-YFP to chloroplasts. Also note that the only difference between construct #6 and #8 is the fluorescent protein fused to CcmO. Empty microcompartments are also formed when CcmO-YFP, CcmK2 and CcmL are simultaneously targeted into chloroplasts (construct #11). These results are shown in FIG. 6.

### Example 9

### N17-CCFP (P35SS), CcmK2 (Pmas), CcmO-YFP (Pug10) (Construct #12)

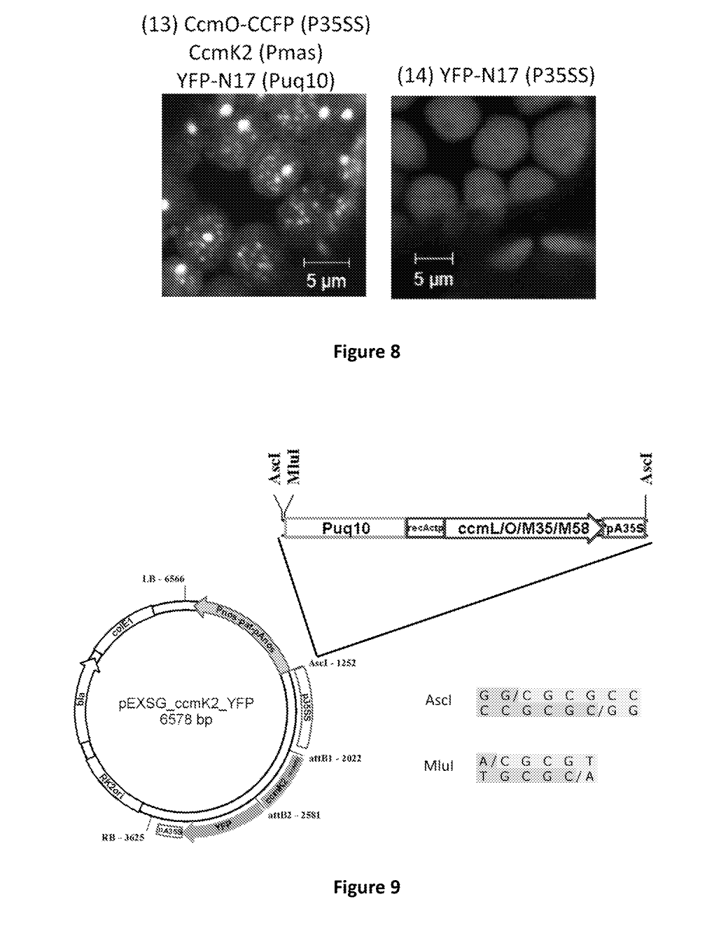

When the 17 AA peptide (N17) is put on the N terminus of a foreign protein (cyan fluorescent protein, CCFP), the protein does not localize with the microcompartments (construct #12). These results are shown in FIG. 7.

### Example 10

### CcmO-CCFP (P35SS) CcmK2 (Pmas) YFP-N17 (Pug10) (Construct #13) and YFP-N17 (P35SS) (Construct #14)

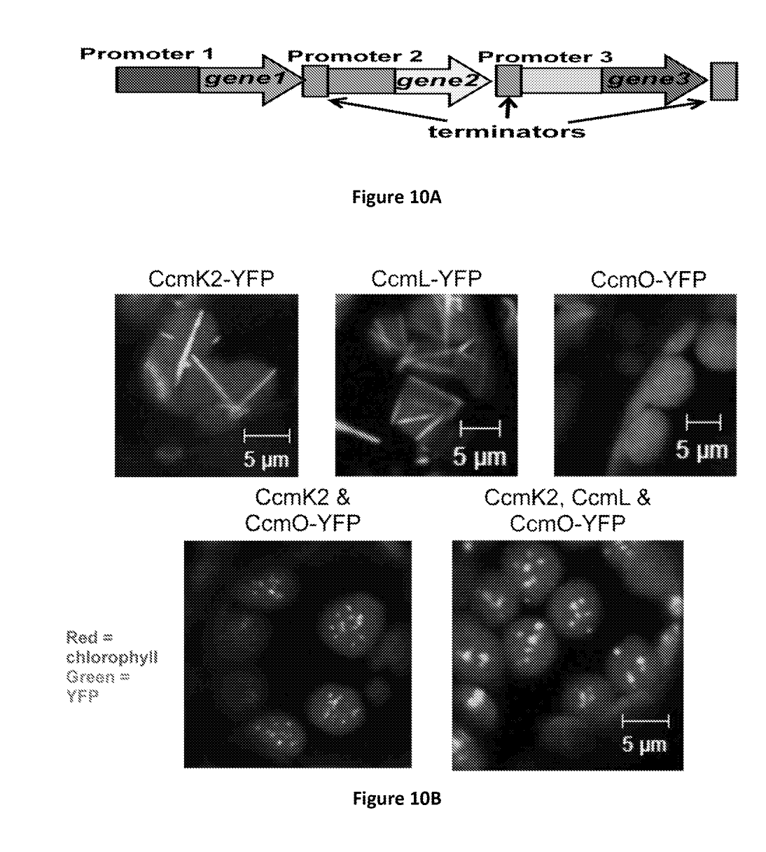

When the signal peptide was used on the C-terminus of YFP (YFP-N17), it co-localizes with compartments formed by CcmK2/CcmO-CCFP (construct #13). YFP-N17 by itself gives diffuse signals only (construct #14). This demonstrates that a foreign protein (YFP) can be targeted to a synthetic compartment with the use of the N17 signal peptide at the C-terminus. These results are shown in FIG. 8.

The results shown in Examples 5-10 demonstrate the following: (1) When CcmK2, CcmL and CcmO are individually targeted to chloroplasts, no microcompartments are formed; (2) the co-expression of CcmK2 and CcmO and CcmM58 (or CcmL) results in the assembly of empty microcompartments only when no fluorescent protein is fused to CcmK2; (3) the co-expression of CcmK2, CcmL, and CcmO also results in the assembly of empty microcompartments when no fluorescent protein is fused to CcmK2 and CcmL; (4) different combinations of promoters can be used to achieve the assembly of empty microcompartments from CcmK2 and CcmO and CcmM5 (or CcmL); (5) this is the first time compartments are synthesized with proteins from a bacterial microcompartment in a eukaryotic cell; (6) the 17-amino-acid signal peptide from CcmN (N17) can be used to target a foreign protein to the microcompartments assembled from CcmK2 and CcmO when the signal peptide is fused to the C-terminus of the foreign protein; and (7) a foreign protein has been successfully targeted to microcompartments assembled with β-carboxysomal proteins.

Summary of results from Examples 1-10: (1) Constructs #1, 2, 3, 4, 5, 7 do not form microcompartments; (2) Constructs #6, 8, 9, 10 likely do not form microcompartments; (3) Constructs #12, 13 and 14 are for the signal peptide N17; Construct #12 is presently inoperable; Construct #13 is operable; and Construct #14 is a control expressing only the signal peptide fused to YFP in the absence of microcompartments.

### Example 11

### Expression Vectors Useful for Expressing Proteins from the Nucleus

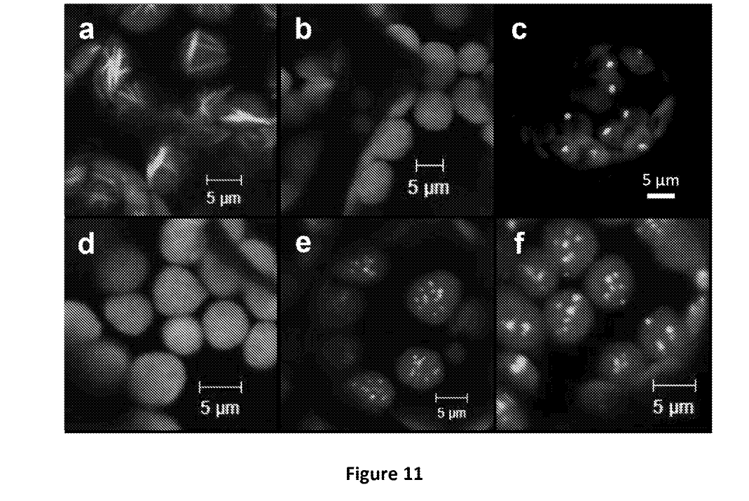

An exemplary vector useful for expressing proteins from the nucleus is shown in FIG. 9.

### Example 12

### Transient Expression by Agroinfiltration

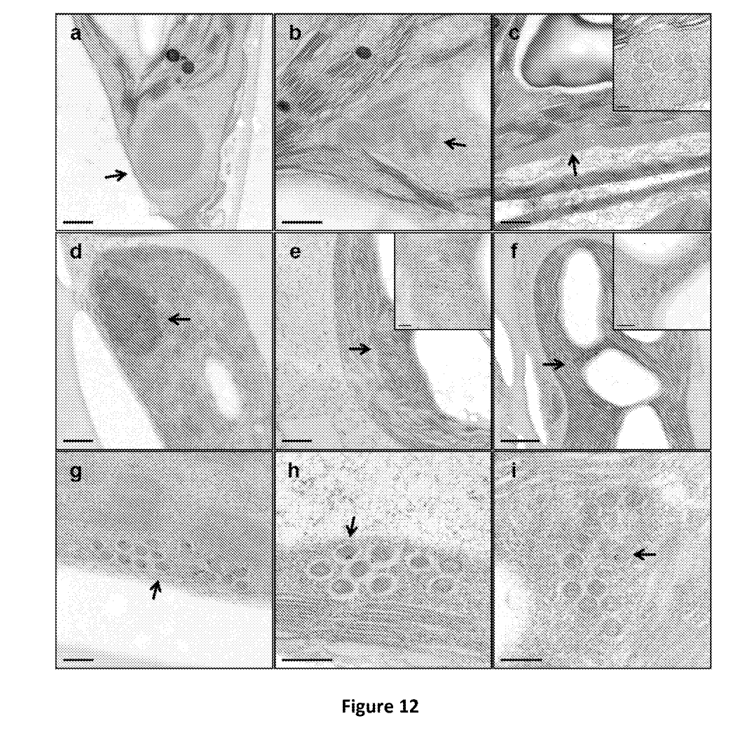

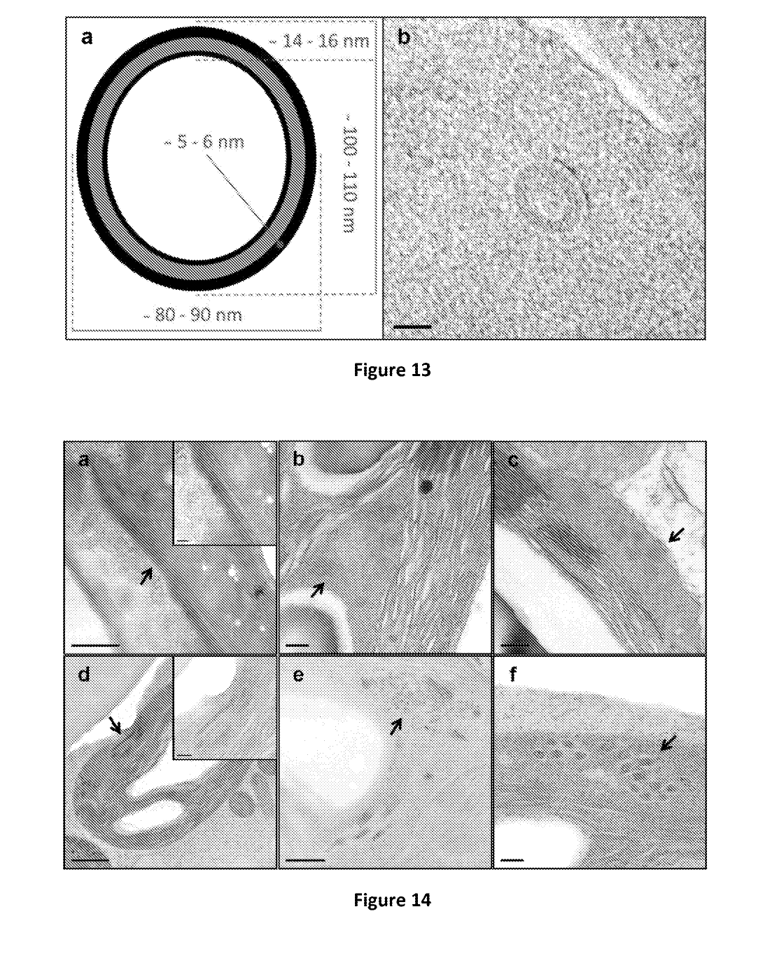

β-carboxysomal proteins are transiently expressed in tobacco and N. benthamiana leaves following agroinfiltration. Each expression vector contains 1-3 transgenes driven by different promoters (FIG. 10A). When expressed individually, CcmK2 and CcmL result in abnormal rod shapes. CcmO signal is diffuse. When they are simultaneously expressed, punctate fluorescent signals are seen. These results are depicted in FIG. 10B.

### Example 13

### Targeting of CcmK2, CcmO-YFP, CcmL (Construct #15), and CcmM58-YFP (Construct #16) Proteins to Chloroplasts

When two agrobacterial strains bearing constructs #15 and #16 (FIG. 2) respectively were co-infiltrated into Benthamiana's leaves, spherical bodies of ˜100 nm were observed in chloroplasts. Larger proteins aggregates were also formed. The spherical bodies have clearly defined outlines and are very uniform in shape and size, highly resembling some kind of microcompartments.

### Example 14

### β-Carboxysomal Proteins Assemble into Highly Organized Structures in Nicotiana Chloroplasts

Using the agroinfiltration technique, in this Example, we have transiently expressed multiple β-carboxysomal proteins (CcmK2, CcmM, CcmL, CcmO and CcmN) in Nicotiana benthamiana with fusions that target these proteins into chloroplasts and that provide fluorescent labels for visualizing the resultant structures. By confocal and electron microscopic analysis, we have observed that the shell proteins of the β-carboxysome are able to assemble in plant chloroplasts into highly organized assemblies resembling empty microcompartments. We demonstrate that a foreign protein can be targeted with a 17-amino-acid CcmN peptide to the shell proteins inside chloroplasts. The results of our experiments are as follows.

Results

All carboxysomal proteins expressed in this Example were fused with the N-terminal chloroplast transit peptide from Arabidopsis recA gene (Kohler et al. Science, 276, 2039-2042, 1997). Imaging analyses indicate that all the proteins were correctly targeted to chloroplast stroma.

Transient Expression of CcmK2-YFP in N. benthamiana Leaves

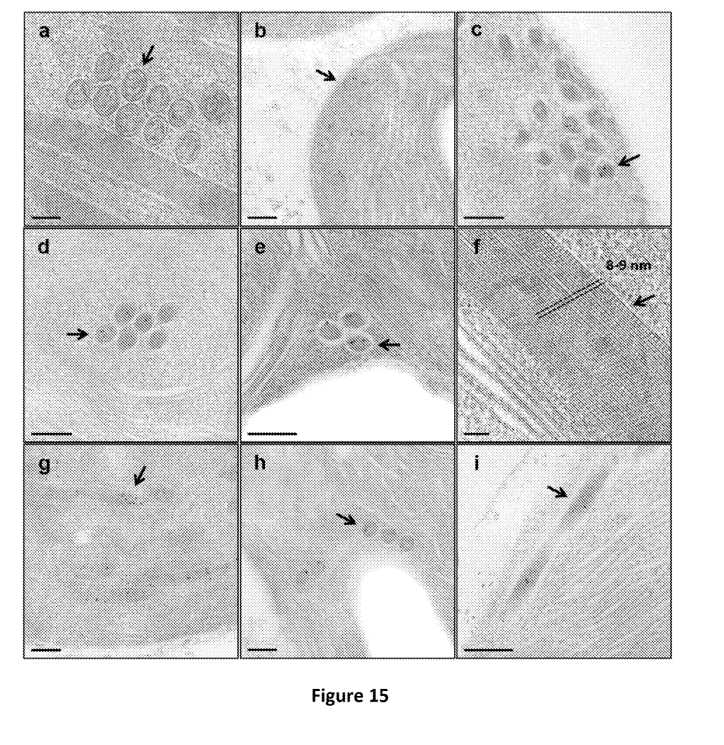

When yellow fluorescent protein (YFP)-tagged CcmK2 (CcmK2-YFP) was expressed and targeted to the chloroplasts of N. benthamiana, elongated structures were visualized by fluorescent microscopy (FIG. 11a). Co-expression of CcmK2-YFP with other carboxysomal proteins did not alter these elongated fluorescent signals. We hypothesize that fusing YFP to the much smaller CcmK2 protein causes the CcmK2 subunits to assemble incorrectly, leading to these elongated structures and preventing proper interactions with other carboxysomal proteins. Hence, subsequent experiments were performed with CcmK2 lacking an YFP tag.

Transient Expression in N. benthamiana Leaves of CcmO, CcmK2, CcmL and CcmM58

When CcmO-YFP is targeted to chloroplasts, diffuse YFP signals were observed; alternatively, in some cases, polar aggregations were seen, probably due to very high protein levels (FIG. 11b,c). We co-expressed CcmO-YFP with each of the other β-carboxysomal shell proteins, namely CcmK2, CcmK3, CcmK4 and CcmL. We found that only in the presence of CcmK2 was CcmO-YFP able to produce punctate fluorescent loci (FIG. 11d,e), indicating the possible assembly of CcmK2 and CcmO-YFP. Punctate fluorescent signals were consistently produced when additional proteins such as CcmL and CcmM58 were co-expressed with CcmK2 and CcmO-YFP (FIG. 11f).

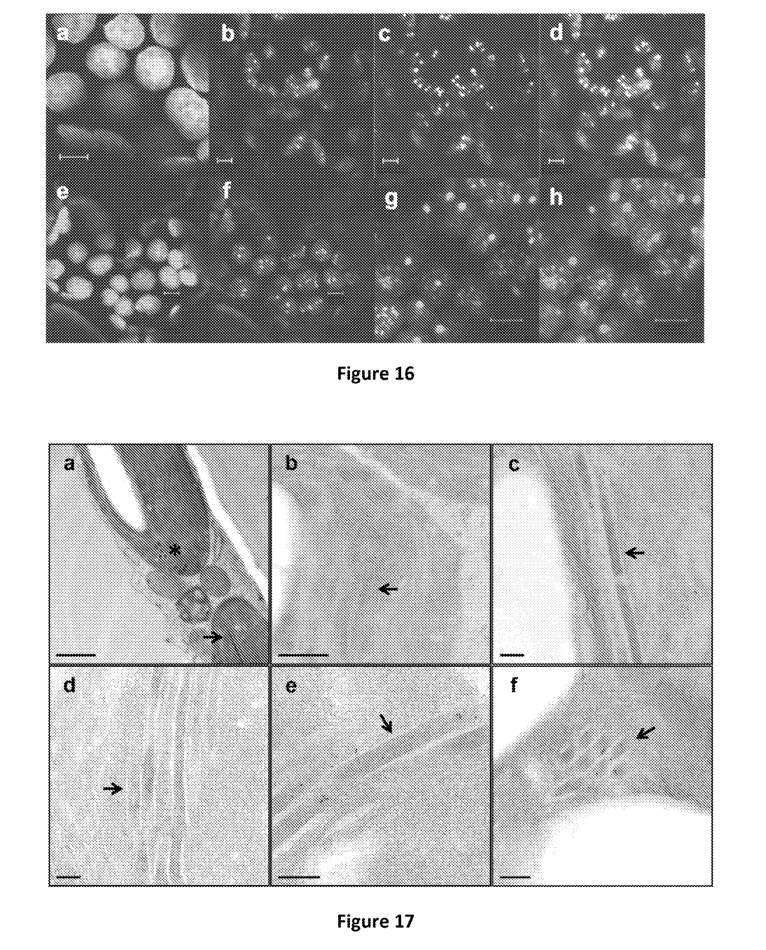

In order to further resolve the structures formed by these punctate signals, the plant material was characterized at high resolution by transmission electron microscopy (TEM). Two different protocols of preparation of plant tissue were used: normal chemical fixation at room temperature; and high pressure freeze fixation (HPF)/freeze substitution in combination with immunogold labelling. In leaves expressing CcmO-YFP alone, large protein aggregates were observed (FIG. 12a, d). Interestingly, when CcmO-YFP was expressed in combination with CcmK2, protein arrays organized into parallel linear structures were observed (FIG. 12b, e). This result indicates that CcmO-YFP is not able to self-assemble into discrete structures, but when CcmK2 is present, the two carboxysomal proteins can interact, forming ordered assemblies—possibly sheets or stacked arrays of carboxysomal facets. Immunogold experiments using an anti-GFP antibody supported the presence of carboxysomal protein in these structures (FIG. 12 due).

When we expressed another component of the shell, CcmL-YFP, along with CcmO-YFP and CcmK2, circular structures were observed (FIG. 12 c,f-i). The same outcome was observed using either form of sample preparation-normal chemical fixation or HPF fixation. Immunogold experiments using anti-GFP (FIG. 12 f,g), anti-CcmK2 (FIG. 12 h) and anti-CcmO (FIG. 12 i) antisera confirmed the presence of CcmO and CcmK2 in these circular structures.

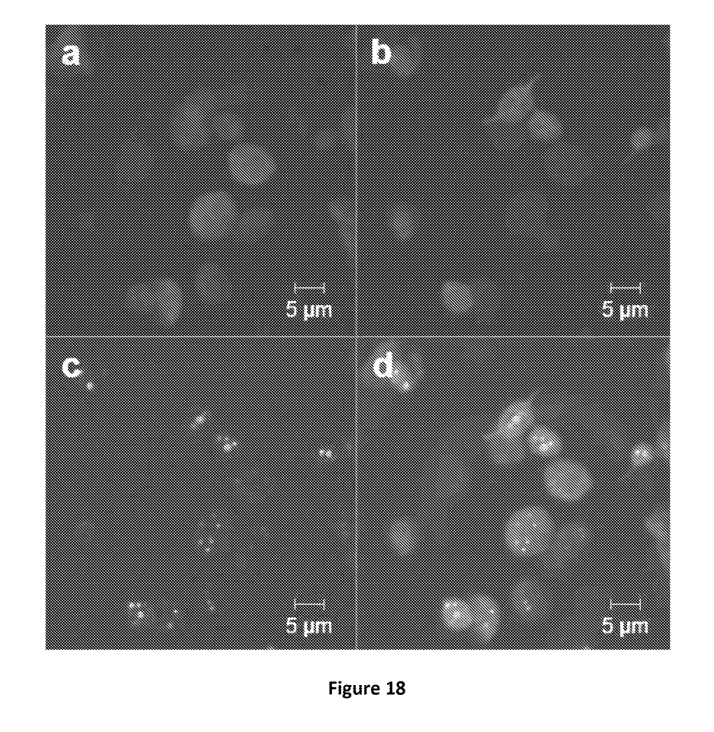

A size determination and a schematic representation of these structures are illustrated in FIG. 13. They are round and slightly elongated, but some angular structures have been observed in high quality plant material fixed by high pressure freezing (FIG. 12 g). They are about 100-110 nm in length and 80-90 nm in width. These carboxysome-like structures are surrounded by a double shell with a space in between. The external shell is about 5-6 nm in thickness, which is a value similar to that reported for a β-carboxysome shell (Kaneko et al. J. Bacteriol., 188, 805-808, 2006). In contrast, the structure of the second shell is difficult to resolve since it appears less thick and disorganized. An inner cavity surrounded by the double shell constitutes the internal part of the circular structure.

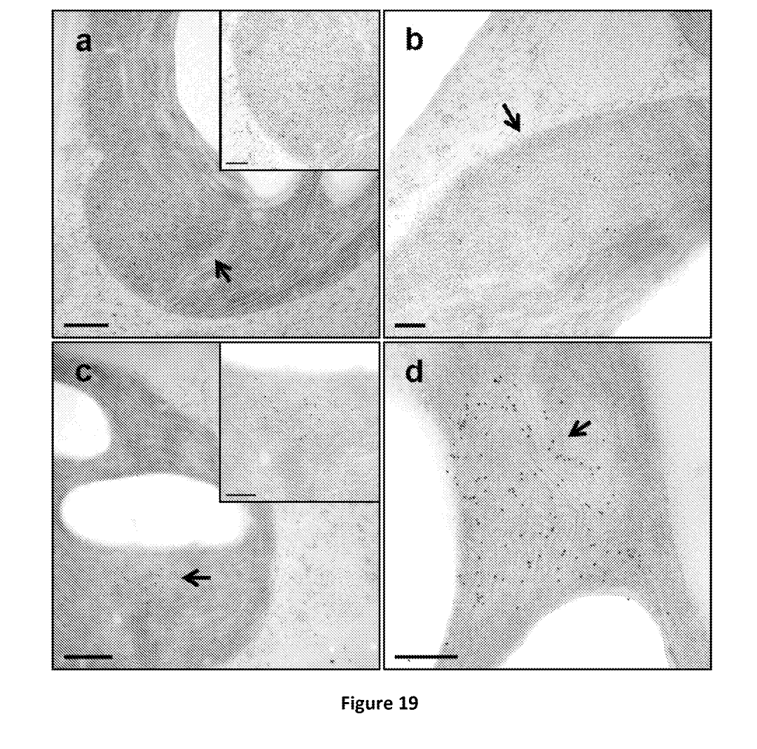

In the same plant material (expressing CcmO-YFP, CcmK2, and CcmL-YFP), other types of structures were observed as well as the round structures described above (FIG. 14). Elongated (FIG. 14 a,d) and disorganized structures (FIG. 14 b,e) were observed, which probably result from different ratios of carboxysomal proteins (CcmO-YFP, CcmK2 and CcmL-YFP), the proportions of which are likely to be heterogeneous within agroinfiltrated leaves. The presence of structures that are intermediate between a round and elongated shape, may also reflect the importance of having an optimal ratio of carboxysomal proteins for carboxysome biogenesis (FIG. 14 c, f).

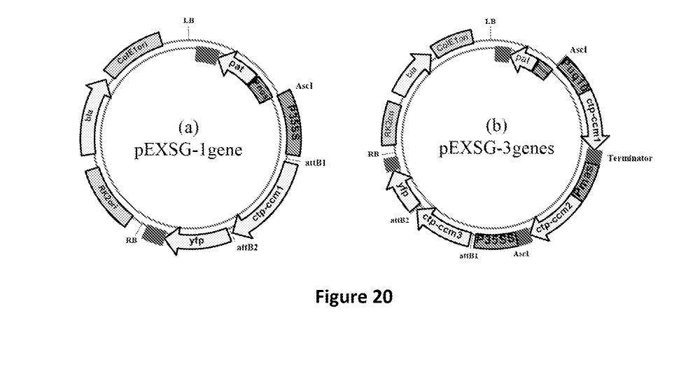

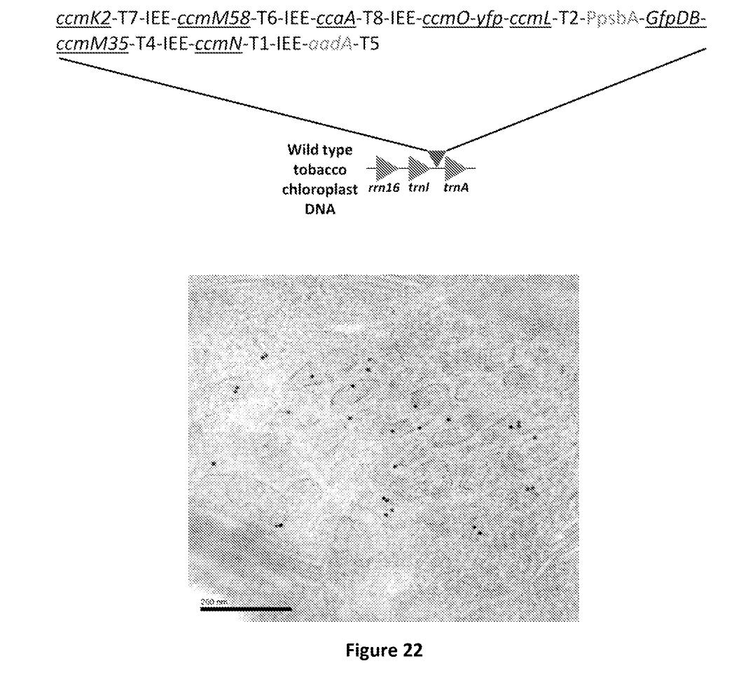

In plant material expressing CcmM58-YFP in combination with CcmO-YFP and CcmK2, round structures were observed (FIG. 15a). Immunogold experiments using antibodies against GFP (FIG. 15b), CcmK2 (FIG. 15c), CcmO (FIG. 15d), and CcmM58 (FIG. 15e) confirmed the presence of all these proteins. This result supports the interpretation that sheets of CcmO-YFP/CcmK2 proteins form complex structures in the presence of CcmM58. In addition to the round structures, elongated structures were observed in some chloroplasts in plants agroinfiltrated with constructs expressing CcmM58-YFP, CcmO-YFP and CcmK2 (FIG. 15f, g). The elongated structures were organized into semicrystalline arrays of parallel lines spaced 8-9 nm, which intersect forming a net structure (FIG. 15f, g). A similar organization of carboxysomal proteins in β-carboxysomes has been described by others (Kaneko et al. J. Bacteriol., 188, 805-808, 2006). Immunogold experiments using antibody against GFP, CcmK2, CcmO and CcmM58 also confirmed the presence of these carboxysomal proteins in elongated structures (FIG. 17).

In plant material expressing CcmO-YFP, CcmK2, CcmL, and CcmM58-YFP together, we observed the same type of carboxysome-like and elongated structures described for the CcmO-YFP, CcmK2, and CcmM58-YFP plant material (FIG. 15h,i). Immunogold experiments with an anti-GFP antibody supported the presence of the carboxysomal proteins.

CcmN Contains a 17-Amino-Acid Peptide that can Target YFP to the Structures Formed by CcmK2/CcmO-YFP

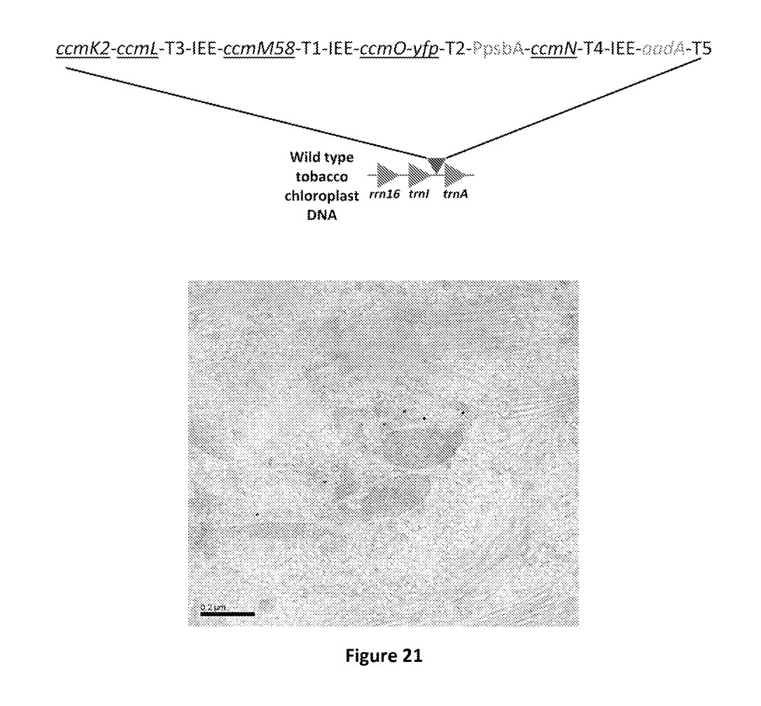

We also co-expressed CcmN, an internal protein of β-carboxysomes, with CcmK2 and CcmO. When expressed alone, CcmN-CFP gave diffuse CFP signals (FIG. 16a). When CcmN-CFP was co-expressed with CcmK2 and CcmO-YFP, both CcmN-CFP and CcmO-YFP gave punctate signals, which co-localized to the same areas in chloroplasts, suggesting that CcmN was able to associate with the structures composed of CcmK2 and CcmO-YFP (FIG. 16b-d). When we removed the C-terminal 17 amino-acid peptide of CcmN and fused the truncated CcmN to CFP, the resulting protein fusion, CcmNd17-CFP, no longer co-localized with the punctate structures of CcmK2 and CcmO-YFP (FIG. 16e, f).

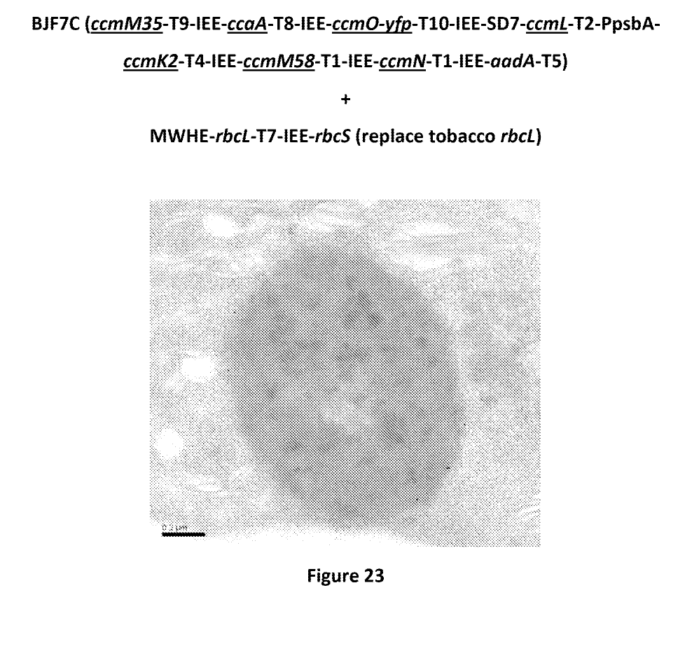

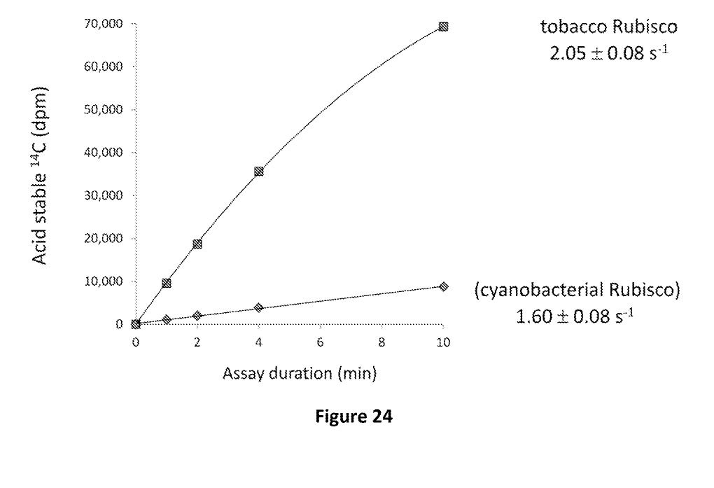

In order to test the ability of the CcmN17 peptide to target a foreign protein to the structures formed by CcmK2 and CcmO-YFP, we co-expressed CcmK2 and CcmO-YFP with CcmN17-CFP, where the CFP was fused to the C-terminus of CcmN17. But the CcmN17-CFP fluorescent signals were diffuse and did not co-localize with the punctate signals of CcmO-YFP (FIG. 18). We hypothesize that either the CFP fused to the C-terminus of CcmN17, or the 15 amino-acid scar peptide that remained at the N-terminus of CcmN17 after the truncation of the chloroplast transit peptide, interfered with the interaction between CcmN17 and CcmK2. When we fused the CTP-YFP to the N-terminus of CcmN17 and co-expressed it with CcmK2 and CcmO-CFP, both YFP-CcmN17 and CcmO-CFP fluorescent signals co-localized to punctate spots within chloroplasts (FIG. 16g-h). Thus, the C-terminal 17 amino-acid peptide of CcmN (CcmN17) is critical for the binding of CcmN to the structures of CcmK2 and CcmO-YFP, and the CcmN17 peptide by itself is enough to target a foreign protein to the shell proteins of β-carboxysomes. However, we were not able to observe by TEM the formation of any specific structures in the leaf tissues expressing CcmN or CcmN17 with CcmK2 and CcmO (FIG. 19).

The above-referenced Results in the Example were obtained using the following method and experimental procedures.

Experimental Procedures

Plant Expression Vector Construction

The Synechococcus elongatus PCC7942 ccmK2, ccmL and ccmO genes were amplified from E. coli expression vectors containing the respective coding regions, kindly provided by Cheryl Kerfeld (Michigan State University). Synthetic ccmN, ccmM58, ccmK3, ccmK4 and yfp genes, designed to mimic the codon usage of chloroplast protein expression system, were synthesized by Bioneer Inc. (Alameda, Calif.). Each carboxysomal gene was fused with the chloroplast transit peptide (ctp) from Arabidopsis recA gene (Kohler, et al. Science, 276, 2039-2042, 1997).

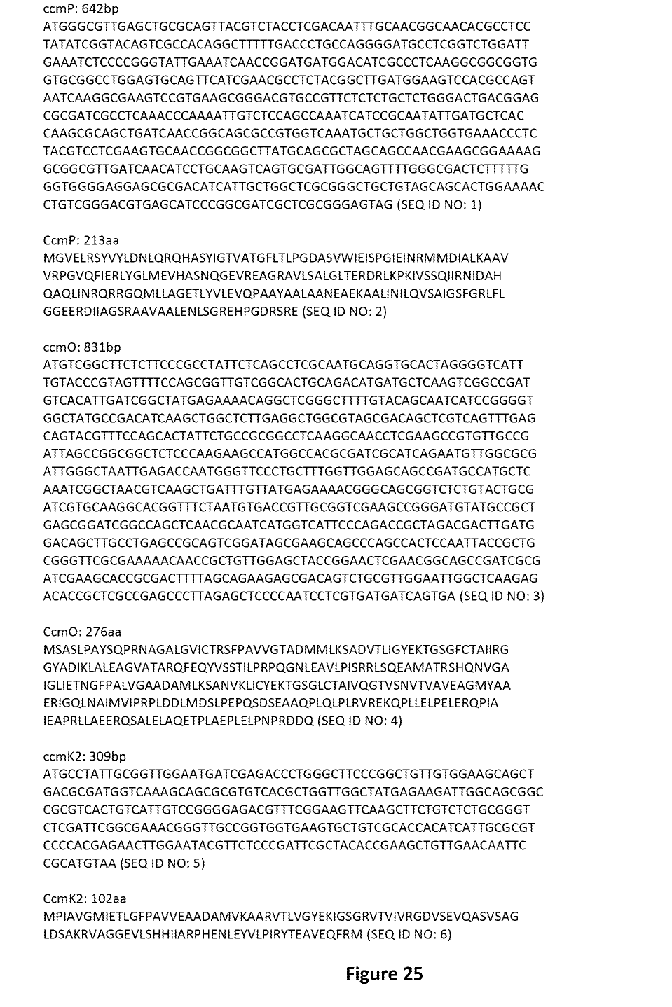

Table 1 (below) contains the primers used in the overlap extension PCR procedure (Horton et al. Gene 77:61-68, 1989) to generate the ctp::ccm gene fusion constructs with Phusion High-Fidelity DNA polymerase (Thermo Scientific). The PCR products were first cloned into pCR8/GW/TOPO TA vector (Life Technologies) and subsequently transferred to the pEXSG-YFP Gateway destination vector, which has the tandem CaMV 35S promoter (P35SS) and the YFP or CFP gene placed 5′ and 3′ of the Gateway recombination cassette respectively (Jakoby et al. Plant Physiol., 141, 1293-1305, 2006), through a standard LR recombination reaction. Thus, each resulting vector contains a carboxysomal gene driven by P35SS and fused to the YFP or CFP gene at the 3′ end as shown in FIG. 20.

TABLE 1
The oligonucleotides used in the construction of plant expression vectors.

| Primers | Sequences | Comments |
|---|---|---|
| CTP-for | ATGGATTCACAGCTAGTCTTGTCTCTG | To amplify the chloroplast |
| CTP-rev | GTCGCGATCGAATTCAGAACTGATTTTGTGGGAG | transit peptide |
| CTP-K2-for | CAAAATCAGTTCTGAATTCGATCGCGACATGCCTATTGCGGTTGGAATGATC | To amplify the ccmK2, ccmL, |
| K2-rev | CATGCGGAATTGTTCAACAGCTTC | ccmO, ccmM58, ccmN, ccmK3, |
| CTP-L-for | CAAAATCAGTTCTGAATTCGATCGCGACATGCGCATTGCTAAGGTTCG | ccmK4 and yfp genes with a |
| L-rev | GCTGTGCTCGCGTTTGTC | 5′ overlap extension to the |
| CTP-O-for | CAAAATCAGTTCTGAATTCGATCGCGACATGTCGGCTTCTCTTCCCGCCT | chloroplast transit peptide |
| O-rev | CTGATCATCACGAGGATTGGGGAG | |
| CTP-M58-for | AGTTCTGAATTCGATCGCGACCCTTCTCCAACAACTGTACCT | |
| M58-rev | CGGCTTCTGAATCAACAACTCA | |
| CTP-N-for | CAAAATCAGTTCTGAATTCGATCGCGACATGCACCTACCACCTCTGGAAC | |
| N-rev | TCGATCAGGGAACATATTTGTCG | |
| CTP-N17-for | CAAAATCAGTTCTGAATTCGATCGCGACTACGGCAAGGAACAGTTTTTGCGGA | |
| Nd17-rev | GACCTTAGTGGGGTGGGCGATTG | |
| CTP-K3-for | CAAAATCAGTTCTGAATTCGATCGCGACATGCCTATTGCTGTAGGTACTATACAAAC | |
| KS-rev | AGATCGGAATGGCTCAGATTCAGC | |
| CTP-K4-for | CAAAATCAGTTCTGAATTCGATCGCGACATGTCTCCAAGCTATTGGATCTCTTG | |
| K4-rev | ACGTCGACCACTACCAGTTCCTTC | |
| CTP-YFP-for | CAAAATCAGTTCTGAATTCGATCGCGACATGGTTAGTAAAGGTGAAGAATTGTTTACTG | |
| YFP-rev | CTTGTACAACTCGTCCATTCCTAAAG | |
| Puq10-for | ATGCACGGCGCGCCTCACACGCGTCGACGAGTCAGTAATAAACGGCGTC | To amplify the ubiquitin-10 |
| Puq10-rev | ACTAGTTCTAGAGGATCCCGCACTCGAGTGTTAATCAGAAAAACTCAGATTAATCGAC | and MAS promoters and 35S |
| Pmas-for | ATGCACGGCGCGCCTCACACGCGTGCACGCCCCAGAGCTTCTC | terminator (AscI and MluI |
| Pmas-rev | GCTAGAGTCGATTTGGTGTATCGAG | restriction sites are |
| pA35S-for | GAGCTCTAGAGTCCGCAAAAATCAC | underlined.) |
| pA35S-rev | ATGCACGTTTAAACGGCGCGCCGGTCACTGGATTTTGGTTTTAGGAATTAG | |
| Puq10-CTP-for | GTCGATTAATCTGAGTTTTTCTGATTAACAGATGGATTCACAGCTAGTCTTGTCTCTG | To amplify CTP fused ccmK2, |
| Pmas-CTP-for | CTCGATACACCAAATCGACTCTAGCATGGATTCACAGCTAGTCTTGTCTCTG | ccmL, ccmO, ccmM58, ccmN, |
| pA35S-K2-rev | GTGATTTTTGCGGACTCTAGAGCTCTTACATGCGGAATTGTTCAACAGCTTC | ccmK3, ccmK4 and yfp genes |
| pA35S-L-rev | GTGATTTTTGCGGACTCTAGAGCTCTTAGCTGTGCTCGCGTTTGTCGTAG | with a 5′ overlap extension |
| pA35S-O-rev | GTGATTTTTGCGGACTCTAGAGCTCTTACTGATCATCACGAGGATTGGGGAG | to the uhiquitin-10 or MAS |
| pA35S-M58-rev | GTGATTTTTGCGGACTCTAGAGCTCTTACGGCTTCTGAATCAACAACTCAGC | promoter and a 3′ overlap |
| pA35S-N-rev | GTGATTTTTGCGGACTCTAGAGCTCTTATCGATCAGGGAACATACTTTGTCGCATC | extension to the 35S |
| pA35S-K3-rev | GTGATTTTTGCGGACTCTAGAGCTCTTAAGATCGGAATGGCTCAGATTCAGC | terminator |
| pA35S-K4-rev | GTGATTTTTGCGGACTCTAGAGCTCTTAACGTCGACCACTACCAGTTCCTTC | |
| pA35S-YFP-rev | GTGATTTTTGCGGACTCTAGAGCTCTTACTTGTACAACTCGTCCATTCC | |
| ggsN17-for | GTTCTGGTGGAAGTGGGGGTTCATACGGCAAGGAACAGTTTTTGCGGA | To amplify ccmN17 and |
| ggsN-for | GGAGGTTCTGGTGGAAGTGGGGGTTCAATGCATCTACCGCCCCTAGAG | ccmNd17 with 3xGGS linker |
| ggsYFP-rev | TGAACCCCCACTTCCACCAGAACCTCCCTTGTACAACTCGTCCATTCCTAAAG | at the 5′ end and yfp with |
| N-stop-rev | TTAGCGATCGGGGAACATGCTC | 3xGGS linker at the 3′ end |
| Nd17-Stop-rev | TTAGACCTTAGTGGGGTGGGCGATTG | |

In order to express 1-2 additional carboxysomal genes from single vectors, nuclear expression operons driven by ubiquitin-10 (Puq10) and MAS (Pmas) promoters (Langridge et al. Proc. Natl. Acad. Sci. U.S.A, 86, 3219-3223, 1989, Grefen et al. Plant J., 64, 355-365, 2010) were constructed with the overlap extension PCR procedure using the primers listed in Table 1. These operons were then inserted into the AscI site located 5′ of the tandem CaMV 35S promoter in the pEXSG-YFP vectors (FIG. 20). The attB2 segment located between the carboxysomal gene and YFP or CFP gene in these vectors gives rise to a 15-amino-acid Gateway linker peptide (KGEFDPAFLYKVVDG) upon translation, which should provide sufficient separation between the carboxysomal protein domains and the fluorescent domain. In operons driven by the ubiquitin-10 or MAS promoter, we added 9- or 12-amino-acid flexible linker peptide made up of GGS repeats between the YFP and carboxysomal proteins in order to minimize the influence of the YFP fusion on the molecular interactions among carboxysomal proteins. The expression vectors created in this Example and their features are summarized in Table 2. The YFP and CFP used in this study are EYFP and mCerulean versions and their amino-acid sequences are included in the supplemental information section.

TABLE 2
The plant expression vectors with β-carboxysomal genes created in this study

| Vectors | Promoters^{#} and proteins to be expressed |
|---|---|
| pEXSG-K2-YFP | P35SS - CcmK2-YFP |
| pEXSG-O-YFP | P35SS - CcmO-YFP |
| pEXSG-L-YFP | P35SS - CcmL-YFP |
| pEXSG-M58-YFP | P35SS - CcmM58-YFP |
| pEXSG-O-YFP -UK2 | P35SS - CcmO-YFP, Puq10 - CcmK2 |
| pEXSG-O-YFP -UL | P35SS - CcmO-YFP, Puq10 - CcmL |
| pEXSG-O-YFP -UK3 | P35SS - CcmO-YFP, Puq10 - CcmK3 |
| pEXSG-O-YFP -UK4 | P35SS - CcmO-YFP, Puq10 - CcmK4 |
| pEXSG-O-YFP -UK2-UL | P35SS - CcmO-YFP, Puq10 - CcmK2, Puq10 - CcmL |
| pEXSG-N-CFP | P35SS - CcmN-CFP |
| pEXSG-N-CFP-UK2-PmO-YFP | P35SS - CcmN-CFP, Puq10 - CcmK2, Pmas - CcmO-YFP |
| pEXSG-Nd17-CFP-UK2-PmO-YFP | P35SS - CcmNd17-CFP, Puq10 - CcmK2, Pmas - CcmO-YFP |
| pEXSG-N17-CFP-PmK2-UO-YFP | P35SS - CcmN17-CFP, Pmas - CcmK2, Puq10 - CcmO-YFP |
| pEXSG-YFP-N17 | P35SS - YFP-CcmN17 |
| pEXSG-O-CFP-PmK2-UYFP-N17 | P35SS - CcmO-CFP, Pmas - CcmK2, Puq10 - YFP-CcmN17 |
| pEXSG-YFP-N17-UO-PmK2 | P35SS - YFP-CcmN17, Puq10 - CcmO, Pmas - CcmK2 |
| pEXSG-YFP-Nd17-UO-PmK2 | P35SS - YFP-CcmNd17, Puq10 - CcmO, Pmas - CcmK2 |

^{#}P35SS - tandem 35S promoter, Puq10 - ubiquitin-10 promoter, Pmas - MAS promoter
Each expression vector was electroporated into Agrobacterium tumefaciens GV3101/pMP90RK (Koncz and Schell Mol. Gen. Genet., 204, 383-396, 1986) and transformants were selected on LB agar plates containing carbenicillin, kanamycin and gentamycin.

Transient Expression of Carboxysomal Proteins in Nicotiana benthamiana

For transient expression of carboxysomal proteins in N. benthamiana leaf tissues, agroinoculation was performed as described previously (Sparkes et al. Nat Protoc, 1, 2019-2025, 2006). About 5 ml of each agrobacterial culture grown to the late log phase was pelleted, re-suspended in 10 mM MES pH 5.6 buffer with 10 mM MgCl2 and 150 uM acetosyringone to an optical density of 0.3-0.5 and incubated in the dark at 28 degree C. for 2-5 h. Leaves from 4-6 week-old N. benthamiana plants grown at 10 hours of light per day at ˜50 μmoles/m2/s of light intensity at around 22 degree C. were infiltrated with the agrobacterial suspension on the abaxial side using a needle-less syringe. In some cases, suspension of agrobacteria carrying the gene for the p19 protein of tomato bushy stunt virus (TBSV) was also co-infiltrated to improve the expression levels and duration of carboxysomal proteins (Voinnet et al. Plant J., 33, 949-956, 2003). Within 2-4 days after agroinfiltration, the leaf tissues were examined with a confocal microscope or fixed for immunogold labelling and transmission electron microscopy as described below.

Confocal Microscopy

Laser scanning confocal microscopy was performed on a Zeiss LSM 710 confocal microscope through a 25× multi-immersion objective. The 458, 488 and 514 nm lines of an argon laser were used to excite CFP, chlorophyll and YFP respectively. All the imaging experiments were carried out in sequential mode in order to minimize cross-talk from undesired fluorophores. The images were collected and processed with either Zen 2009 or 2010 microscope software (Carl Zeiss Microscopy, Jena, Germany).

Chemical Fixation, Dehydration, and Embedding

2 mm pieces of leaf tissue were taken using a razor blade and then incubated in primary fixative (2.5% glutaraldehyde, 4% paraformaldehyde in 0.05M phosphate buffer pH 7.2) for 2 hours in low vacuum, over ice with rotation. The samples were washed three times in 0.05M phosphate buffer pH 7.2 and then incubated in the second fixative (1% osmium tetroxide in 0.05M phosphate buffer pH 7.2) for 4 hours over ice with rotation. Then the tissue was washed again in 0.05M phosphate buffer pH 7.2 and dehydrated through an acetone series at room temperature. This step was performed washing the samples in 30%-50%-70%-90% (v/v) acetone in water (10 minutes each incubation) and then three times in 100% dry acetone (30 minutes each incubation). Finally the samples were embedded through increasing concentration of Spürr resin (TAAB) from 30%-50%-70% to 100% v/v of resin in acetone and then polymerized overnight at 60° C. (Hulskamp et al. Cold Spring Harbor protocols, 2010, pdb prot4958, 2010).

Cryofixation Using a High Pressure Freezer, Freeze Substitution, and Embedding

5 mm disks of leaf tissue were taken using a punch and then incubated in 150 mM sucrose for 8 minutes in low vacuum, in order to fill the intercellular spaces. The disks were transferred in the cavity of a specimen carries (type B planchettes, Leica Microsystems) coated with 1-hexadecene and containing 150 mM sucrose as cry-protectant. The flat side of a second carries was used as a lead. This plant material was then cryo-fixed using a high pressure freezer unit (Leica Microsystems EM HPM100).

For the second step of freeze substitution, the high pressure frozen tissue was transferred in small containers pre-cooled in liquid nitrogen and containing a solution of 0.5% uranyl acetate in dry acetone. The freeze substitution was carried out in an EM AFS unit (Leica Microsystems) at −85° C. for 48 hours and then linear warm up to −60° C. for 5 hours. After 1 hour wash at −60° C. using dry ethanol, the samples were infiltrated at low temperature in Lowicryl HM20 resin (Polysciences). This step was performed at −60° C. through increasing concentration of resin, from 30%-50%-70% to 100% v/v HM20 in dry ethanol, for 1 hour each incubation. Finally the samples were transferred in aluminium moulds and polymerized at −50° C. for 24 hours using an UV lamp (Hillmer et al. J. Microsc., 247, 43-47, 2012).

Immunogold Labelling

Gold grids carrying ultrathin sections (60-90 nm) of plant material embedded in HM20 were incubated in blocking solution (1% w/v BSA in PBS buffer) for 1 hour and then treated for 1 hour with a blocking solution containing the primary antibody. Different primary antibodies against GFP (abcam, rabbit polyclonal to GFP) and different carboxysomal proteins were tested: rabbit polyclonal antibodies against CcmK2, CcmO, CcmM, CcmN and cyanobacterial Rubisco (produced by CRB, Cambridge Research Biochemicals). The grids carrying the sections were washed three times in blocking solution and then incubated for 1 hour in blocking solution containing a secondary antibody conjugated with 10 nm gold particles (abcam, goat polyclonal antibody to rabbit IgG, 10 nm gold conjugated). The excess of secondary antibody was removed washing several times in blocking solution and then washing several times in distilled water.

Transmission Electron Microscopy

Grids carrying ultrathin sections of both embedded samples, cryo-fixed and chemically fixed, were post strained using aqueous solution of uranyl acetate and lead citrate. Images were obtained using a transmission electron microscope Jeol 2011 F operating at 200 kV, equipped with a Gatan Ultrascan CCD camera and a Gatan Dual Vision CCD camera.

Summary

In this Example, we used agroinfiltration technique to express transiently several protein components of the β-carboxysome in chloroplasts of N. benthamiana. We fused several of these proteins with YFP or CFP, which allowed us to monitor the formation of protein assemblies at the resolution of visible light. We found that fusing YFP to a much smaller shell protein of β-carboxysome, CcmK2, gave rise to elongated structures and co-expressing it with other shell proteins did not seem to alter these elongated structures. CcmK2 is the major shell component of β-carboxysomes and has a high tendency to self-polymerize. YFP fusion likely causes distortion in the assembly of CcmK2 subunits, leading to the artificial elongated structures, and prevents CcmK2 from proper interactions with other carboxysomal proteins.

Large protein aggregates, probably due to non-specific interactions, were observed in agroinfiltrated N. benthamiana leaves expressing the fluorescently tagged carboxysome shell protein, CcmO-YFP, while co-expression of CcmK2 and CcmO-YFP gave rise to more organized, parallel structures. CcmO and CcmK2 are presumably the main components in the formation of the faces of the carboxysome shell (Rae et al. Plos One, 7, e43871, 2012). Evidently CcmO-YFP alone is not able to self-assemble in a recognizable fashion, but in the presence of CcmK2, the two proteins can polymerize to form sheets which could represent arrays of shell faces.

When we added CcmL-YFP, the component involved in the formation of vertices of carboxysome shells, round structures resembling carboxysome shells were observed. This result supports the notion that CcmL provides the requisite curvature for the formation of the icosahedral structure of the carboxysome. Circular structures that we observed in chloroplasts appear to be defined by a double shell, which contains an internal cavity. The presence of a double shell represents a thermodynamically stable conformation of a combination of CcmO-YFP, CcmK2, CcmL-YFP in the absence of the other internal components such as cyanobacterial Rubisco.

These carboxysome-like structures are more frequently round and smaller then a normal β-carboxysome shell, but in some cases, internal angular structures have been observed in particularly well preserved plant preparations observed using the HPF fixation technique (FIG. 12g). The round structures measure about 100-110 nm in length and 80-90 nm in width, whereas the diameter of a β-carboxysome from Synechococcus PCC7942 is ˜175 nm. The smaller size and less-angular architecture of the outer shell are probably due to the absence of other important components of the shell and of the carboxysome interior. The spherical structures observed in this study are more uniform in size and appear more organized likely due to the presence of additional components such as CcmO and CcmM58.

In N. benthamiana leaves expressing the three carboxysomal proteins CcmO-YFP, CcmK2, and CcmL-YFP, elongated structures and disorganized structures (FIGS. 15h-i and 17d-f) were also observed. These are probably the result of different ratios of the three carboxysomal proteins. The amount of each protein expressed transiently from T-DNA will vary in different cells, depending on how much T-DNA from each strain enters the cell as well as how much RNA is transcribed and translated. Furthermore, different carboxysomal proteins may vary in stability. We speculate that elongated structures are due to the result of CcmO-YFP and CcmK2 polymerization in the presence of sub-optimal amounts of CcmL. In contrast, perhaps when CcmL is present at an appropriate concentration, carboxysome-like structures can be formed.

Interestingly, we could not detect either round structures or elongated assemblies in the samples co-expressing CcmN or CcmN17 with CcmK2 and CcmO. Although the fluorescence fusions indicated that CcmN and CcmN17 are able to co-localize with these shell proteins, the lack of formation of any specific structure suggests that CcmN by self cannot arrange the shell components into more organized structures without CcmM58 or CcmL.

We have also showed that CcmN was able to associate with co-localize with CcmK2 and CcmO-YFP in chloroplasts through the same C-terminal peptide (CcmN17). In addition, our work also demonstrated that the CcmN17 signal sequence alone is able to cause co-localization of a foreign protein, YFP, with the carboxysomal shell proteins, CcmK2 and CcmO, inside the chloroplasts. By expressing CcmO-YFP and CcmK2 in combination with CcmL-YFP and/or CcmM58-YFP, we observed discrete structures with characteristics similar to β-carboxysomes. Therefore, this work provides evidence that specific combinations of carboxysomal proteins can assemble within the chloroplast stroma. These experiments are the first step towards the expression of a complete cyanobacterial carboxysome-based carbon-concentrating mechanism in vascular plants, in order to enhance photosynthetic performance.

### Example 15

### Additional Characterization of Chloroplast Transformants Expressing Carboxysomal Proteins

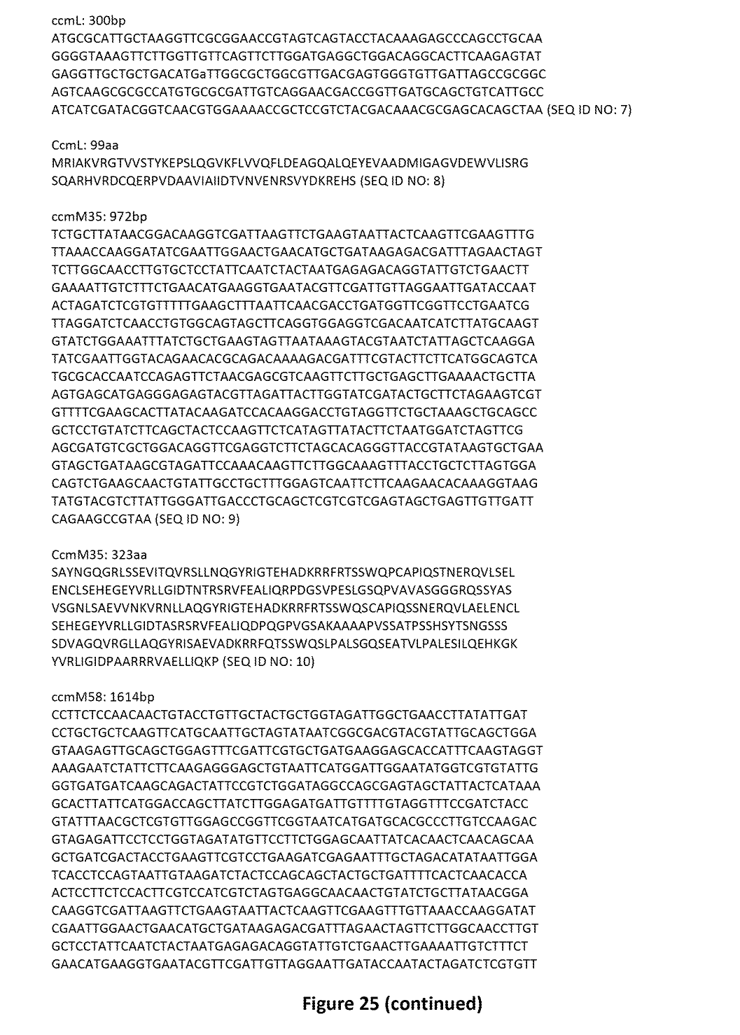

TEM images/YFP immunogold of tobacco lines expressing the following carboxysomal proteins (a) CcmK2, CcmO-YFP, CcmL, CcmM58, and CcmN, (b) CcmK2, CcmO-YFP, CcmL, CcmM58, CcmM35, CcmN, and carbonic anhydrase, and (c) CcmK2, CcmO-YFP, CcmL, CcmM58, CcmM35, CcmN, CcaA (carbonic anhydrase), and cyanobacterial RuBisco are shown respectively in FIGS. 21, 22, and 23. Microcompartment dimensions were found to range from approximately 50 nm to 300 nm. Characterization by immunolabeling demonstrates that carboxysomal proteins are assembled, in tobacco chloroplasts, forming microcompartments.

### Example 16

### RuBP-Dependent ¹⁴CO₂ Fixation by Crude Leaf Homogenates from Homoplastomic Tobacco Lines Expressing Cyanobacterial Rubisco

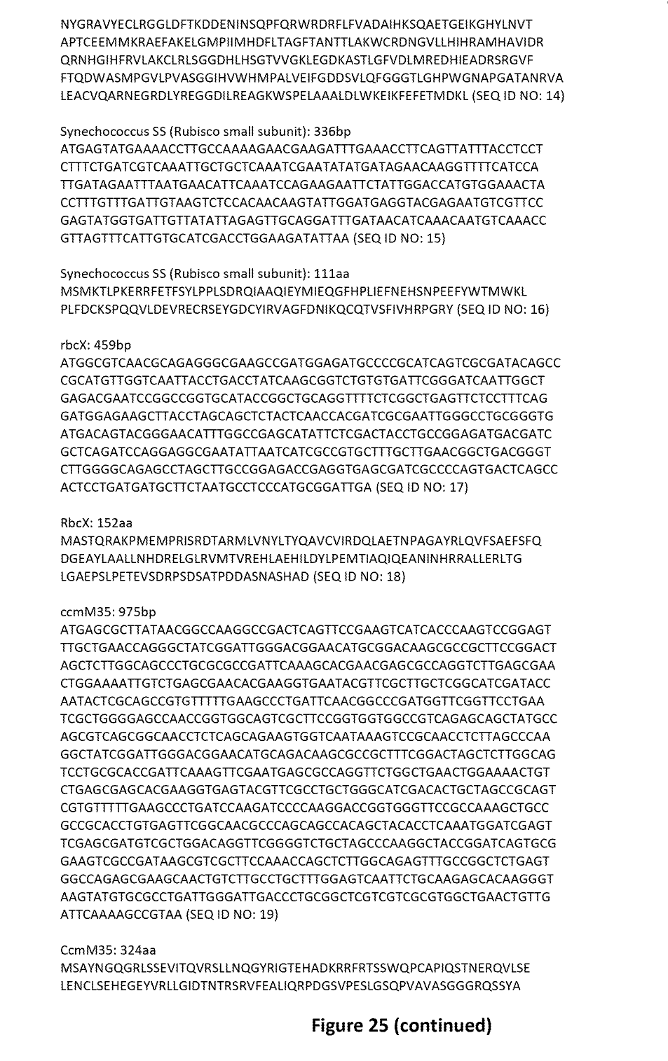

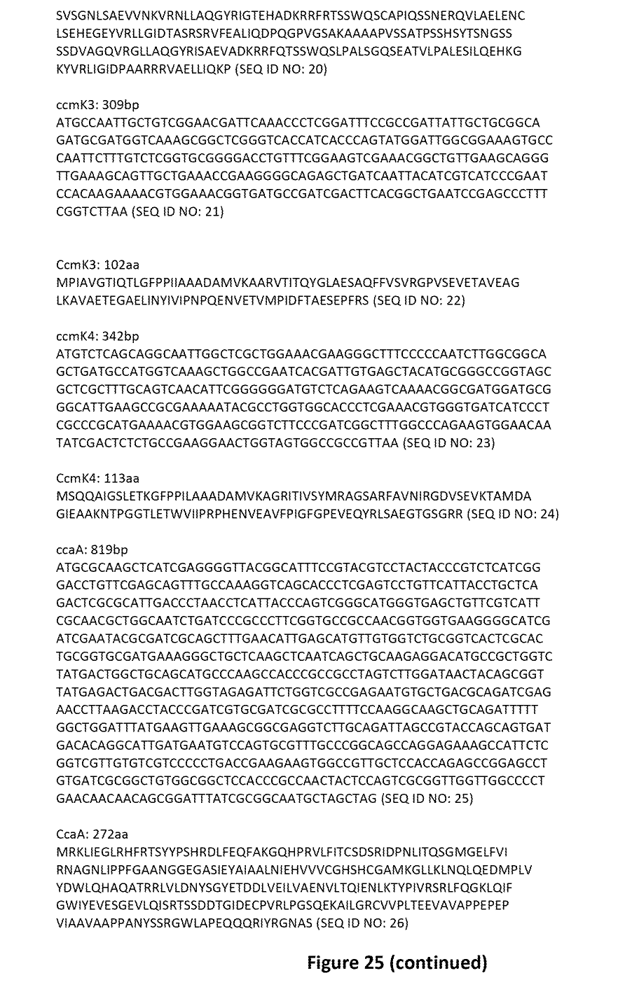

Chloroplast transformants were generated by replacing the tobacco rbcL gene with the cyanobacterial genes shown in FIG. 23. A homoplastomic tobacco line expressing cyanobacterial Rubisco was found to grow at 1% of atmospheric CO₂ according to standard methods. As is shown in FIG. 24, cycanobacterial Rubisco present in crude leaf homogenates showed clear enzymatic activity (RuBP-dependent CO₂ fixation).

### Uses

A number of applications of the microcompartments are described herein. For example, biosynthetic or metabolic pathways may be protected against inhibitors, toxic molecules encapsulated to protect the cell, and valuable proteins or compounds may be sequestered into structures readily purified from cell extracts. A number biomedical applications may also be employed using the disclosed microcompartments, for example, encapsulating one or more vaccines.

Incorporating microcompartments such as carboxysomes into chloroplasts improves photosynthesis by reducing photorespiration. Improvement of photosynthesis accordingly results in increased yield of agricultural products. In addition, being able to produce microcompartments in the plant cell cytoplasm or chloroplasts is also beneficial for other biotechnological applications. Furthermore, strategies disclosed herein for production of microcompartments in plant cells is readily applicable to other types of eukaryotic cells.

In addition, proteins of interests that can be co-localized with the synthetic microcompartments produced from β-carboxysomal proteins, with the use of the signal peptide and linking structures, include, but are not limited to, industrial enzymes such as cellulose-degrading enzymes, oxygen-sensitive enzymes such as nitrogenase or Rubisco, or pharmaceutical proteins that need to be protected from the cellular or chloroplast stromal environment for optimum activity and folding. Enzymes may be incorporated into the microcompartments along with transporters for their substrates and products to improve reaction efficiency. The synthetic microcompartments and co-localized proteins may be used to improve photosynthesis (by protection from oxygen through concentration of carbon dioxide near Rubisco), or to synthesize compounds that would be harmful to the cell if not sequestered in microcompartments, to introduce nitrogen fixation into new tissues, to improve purification of valuable proteins by using microcompartment isolation as a step in the purification process, to produce biosynthetic products that require sequestration of enzymes and reactants. These applications are not limited to plant cells, but may also be utilized in a variety of cells such as in algae, bacteria, fungi, insect and mammalian cells.

### Other Embodiments

All publications mentioned in the above specification are hereby incorporated by reference. Various modifications and variations of the described methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A vascular plant comprising recombinant microcompartments.

2. The plant of claim 1, wherein the microcompartments are round and slightly elongated.

3. The plant of claim 1, wherein the microcompartments are about 100-110 nm in length and about 80-90 nm in width.

4. The plant of claim 1, wherein the microcompartments are about 1-3 μm in length and about 1-3 μm in width.

5. The plant of claim 1, wherein the microcompartments are in chloroplasts of the plant.

6. The plant of claim 1, wherein the microcompartments are in the cytoplasm of the plant.

7. The plant of claim 1, wherein the plant comprises non-naturally occurring expression constructs expressing at least one microcompartment gene stably integrated in the chloroplasts of the plant.

8. The plant of claim 1, wherein the plant is a C3 plant.

9. The plant of claim 1, wherein said microcompartments comprise a protein substantially identical to CcmO, a protein substantially identical to CcmK2, and a protein substantially identical to CcmL.

10. The plant of claim 1, wherein said microcompartments comprise a protein substantially identical to CcmO, a protein substantially identical to CcmK2, and a protein substantially identical to CcmM58.

11. The plant of claim 9 or 10, wherein said microcompartments further comprise a protein substantially identical to CcmM35 or a protein substantially identical to rbcX or a protein substantially identical to CcmN.

12. The plant of claim 11, wherein said microcompartments further comprise a protein having substantial identity to a cyanobacterial ribulose bisphosphate carboxylase large subunit or a cyanobacterial ribulose bisphosphate carboxylase small subunit or both.

13. The plant of claim 12, wherein said microcompartments further comprise a protein having substantial identity to a carbonic anhydrase (CcaA).

14. The plant of claim 1, wherein said microcompartment comprises a protein having substantial identity to CcmK2, a protein having substantial identity to CcmL, a protein having substantial identity to CcmO, a protein having substantial identity to CcmN, a protein having substantial identity to CcmM58, a protein having substantial identity to CcmM35, a protein having substantial identity to CcaA, a protein having substantial identity to cyanobacterial ribulose bisphosphate carboxylase large subunit, and a protein having substantial identity to cyanobacterial ribulose bisphosphate carboxylase small subunit.

15. The plant of claim 14, wherein said microcompartment comprises CcmK2, CcmL, CcmO, CcmN, CcmM58, CcmM35, CcaA, a cyanobacterial ribulose bisphosphate carboxylase large subunit, and a cyanobacterial ribulose bisphosphate carboxylase small subunit.

16. A method of producing a vascular plant having recombinant microcompartments, said method comprising expressing in said plant a protein substantially identical to CcmO, a protein substantially identical to CcmK2, and a protein substantially identical to CcmL or substantially identical to CcmM58, wherein expression of said proteins results in production of recombinant microcompartments in said plant.

17. The method of claim 16, wherein the third protein is substantially identical to CcmL.

18. The method of claim 16, wherein the third protein is substantially identical to CcmM58.

19. The method of claim 16, wherein said plant further expresses a protein substantially identical to CcmM35 or a protein substantially identical to rbcX or a protein substantially identical to CcmN.

20. The method of claim 19, wherein said plant further expresses a protein substantially identical to a cyanobacterial ribulose bisphosphate carboxylase large subunit or a cyanobacterial ribulose bisphosphate carboxylase small subunit or both.

21. The method of claim 20, wherein said plant further expresses a protein substantially identical to carbonic anhydrase (CcaA).

22. The method of claim 16, wherein said microcompartments are round and slightly elongated.

23. The method of claim 16, wherein the microcompartments are about 100-110 nm in length and about 80-90 nm in width.

24. The method of claim 16, wherein the microcompartments are about 1-3 μm in length and about 1-3 μm in width.

25. The method of claim 16, wherein the microcompartments are in chloroplasts of the plant.

26. The method of claim 16, wherein the microcompartments are in the cytoplasm of the plant.

27. The plant of claim 16, wherein the plant comprises non-naturally occurring expression constructs expressing at least one microcompartment gene stably integrated in the chloroplasts of the plant.

28. The method of claim 16, wherein the plant is a C3 plant.

29. The method of claim 16, wherein said plant expresses a protein having substantial identity to CcmK2, a protein having substantial identity to CcmL, a protein having substantial identity to CcmO, a protein having substantial identity to CcmN, a protein having substantial identity to CcmM58, a protein having substantial identity to CcmM35, a protein having substantial identity to CcaA, a protein having substantial identity to cyanobacterial ribulose bisphosphate carboxylase large subunit, and a protein having substantial identity to cyanobacterial ribulose bisphosphate carboxylase small subunit.

30. The method of claim 29, wherein said plant expresses a CcmK2, CcmL, CcmO, CcmN, CcmM58, CcmM35, CcaA, a cyanobacterial ribulose bisphosphate carboxylase large subunit, and a cyanobacterial ribulose bisphosphate carboxylase small subunit.

31. A non-human eukaryotic organism comprising recombinant microcompartments.

32. The organism of claim 31, wherein said organism is a plant, a yeast, a mammal, a fungus, or an insect.

33. The organism of claim 31, wherein the microcompartments are round and slightly elongated.

34. The organism of claim 31, wherein the microcompartments are about 100-110 nm in length and 80-90 nm in width.

35. The organism of claim 31, wherein the microcompartments are about 1-3 μm in length and about 1-3 μm in width.

36. The organism of claim 31, wherein said microcompartments comprise a protein substantially identical to CcmO, a protein substantially identical to CcmK2, and a protein substantially identical to CcmL or to CcmM58.

37. The organism of claim 36, wherein the protein is substantially identical to CcmL.

38. The organism of claim 36, wherein the protein is substantially identical to CcmM58.

39. The organism of claim 36, wherein said microcompartments further comprise a protein substantially identical to CcmM35 or a protein substantially identical to rbcX or a protein substantially identical to CcmN.

40. The organism of claim 39, wherein said microcompartments further comprise a protein substantially identical to a cyanobacterial ribulose bisphosphate carboxylase large subunit or to a cyanobacterial ribulose bisphosphate carboxylase small subunit or both.

41. The organism of claim 40, wherein said microcompartments further comprise a protein substantially identical to a carbonic anhydrase (CcaA).

42. The organism of claim 31, wherein said microcompartments are located in the cytoplasm.

43. The organism of claim 32, wherein said microcompartments are located in plastids of said plant.

44. The organism of claim 32, wherein said microcompartments are located in the cytoplasm of said organisms.

45. A cell comprising recombinant microcompartments.

46. The cell of claim 45, wherein said cell is a bacterial cell, a yeast cell, an insect cell, a plant cell, or a mammalian cell.

47. The cell of claim 45, wherein the microcompartments are round and slightly elongated.

48. The cell of claim 45, wherein the microcompartments are about 100-110 nm in length and 80-90 nm in width.

49. The cell of claim 45, wherein the microcompartments are about 1-3 μm in length and about 1-3 μm in width.

50. The cell of claim 45, wherein said microcompartments comprise a first protein substantially identical to CcmO, a second protein substantially identical to CcmK2, and a third protein substantially identical to CcmL or to CcmM58.

51. The cell of claim 50, wherein the protein is substantially identical to CcmL.

52. The cell of claim 50, wherein the protein is substantially identical to CcmM58.

53. The cell of claim 50, wherein said microcompartments further comprise a protein substantially identical to CcmM58 or a protein substantially identical to CcmM35 or a protein substantially identical to rbcX or to a protein substantially identical to CcmN.

54. The cell of claim 53, wherein said microcompartments further comprise a protein substantially identical to a cyanobacterial ribulose bisphosphate carboxylase large subunit or a cyanobacterial ribulose bisphosphate carboxylase small subunit or both.

55. The cell of claim 54, wherein said microcompartments further comprise an eighth protein substantially identical to a carbonic anhydrase (CcaA).

56. The cell of claim 45, wherein said microcompartments are located in the cytoplasm.

57. The cell of claim 46, wherein said microcompartments are located in plastids of said plant.

58. The cell of claim 46, wherein said microcompartments are located in the cytoplasm of said plant cells.

59. A non-naturally occurring expression cassette comprising a nucleotide sequence encoding at least one of the following:
(i) a protein substantially identical to CcmO;
(ii) a protein substantially identical to CcmK2;
(iii) a protein substantially identical to CcmL;
(iv) a protein substantially identical to CcmN;
(v) a protein substantially identical to CcmM58;
(vi) a protein substantially identical to CcmM35;
(vii) a protein substantially identical to rbcX;
(viii) a protein substantially identical to cyanobacterial ribulose bisphosphate carboxylase rbcL;
(ix) a protein substantially identical to cyanobacterial ribulose bisphosphate carboxylase rbcS;
(x) a protein substantially identical to carbonic anhydrase CcaA; or any combination thereof.

60. The cassette of claim 59, wherein said cassette co-expresses CcmK2 protein, CcmL protein, and CcmO protein.

61. The cassette of claim 59, wherein said cassette co-expresses CcmK2 protein, CcmL protein, CcmO protein, and CcmM58 protein.

62. The cassette of claim 59, wherein said cassette expresses CcmK2 protein.

63. The cassette of claim 59, wherein said cassette expresses CcmL protein.

64. The cassette of claim 59, wherein said cassette expresses CcmO protein.

65. The cassette of claim 59, wherein said cassette expresses CcmM58.

66. The cassette of claim 59, wherein said cassette is stably integrated into a nuclear genome upon transformation into a host cell.

67. The cassette of claim 59, wherein said cassette is stably integrated into a chloroplast genome upon transformation into a host cell.

68. A cell comprising in its genome at least one stably incorporated expression cassette according to any one of claims 59-67.

69. The cell of claim 68, wherein said cell is a plant cell.

70. The cell of claim 69, wherein said expression cassette is stably incorporated in the nuclear genome of the plant cell.

71. The cell of claim 69, wherein said expression cassette is stably incorporated into the chloroplast genome of the plant cell.
